SEQUENCE LISTING (57 sequences)

SEQ ID NO: 1 - protein, 505 aa, Homo sapiens
MDPGNENSATEAAAIIDLDPDFEPQSRPRSCTWPLPRPEIANQPSEPPEVEPDLGEKVHTEGRSEPILLPSRLPEPAGGPQPGILGAVTGPRKGGSRRNAWGNQSYAELISQAIESAPEKRLTLAQIYEWMVRTVPYFKDKGDSNSSAGWKNSIRHNLSLHSKFIKVHNEATGKSSWWMLNPEGGKSGKAPRRRAASMDSSSKLLRGRSKAPKKKPSVLPAPPEGATPTSPVGHFAKWSGSPCSRNREEADMWTTFRPRSSSNASSVSTRLSPLRPESEVLAEEIPASVSSYAGGVPPTLNEGLELLDGLNLTSSHSLLSRSGLSGFSLQHPGVTGPLHTYSSSLFSPAEGPLSAGEGCFSSSQALEALLTSDTPPPPADVLMTQVDPILSQAPTLLLLGGLPSSSKLATGVGLCPKPLEAPGPSSLVPTLSMIAPPPVMASAPIPKALGTPVLTPPTEAASQDRMPQDLDLDMYMENLECDMDNIISDLMDEGEGLDFNFEPDP

SEQ ID NO: 2 - protein, 119 aa, Homo sapiens
ILGAVTGPRKGGSRRNAWGNQSYAELISQAIESAPEKRLTLAQIYEWMVRTVPYFKDKGDSNSSAGWKNSIRHNLSLHSKFIKVHNEATGKSSWWMLNPEGGKSGKAPRRRAASMDSSS

SEQ ID NO: 3 - protein, 39 aa, Homo sapiens
PQDLDLDMYMENLECDMDNIISDLMDEGEGLDFNFEPDP

SEQ ID NO: 4 - protein, 16 aa, Artificial Sequence
  Synthetic Construct
SRRNAWGNQSYAELIS

SEQ ID NO: 5 - protein, 34 aa, Artificial Sequence
  Synthetic Construct
PRKGGSRRNAWGNQSYAELISQAIESAPEKRLTL

SEQ ID NO: 6 - protein, 21 aa, Artificial Sequence
  Synthetic Construct
LECDMDNIISDLMDEGEGLDF

SEQ ID NO: 7 - protein, 33 aa, Artificial Sequence
  Synthetic Construct
PQDLDLDMYMENLECDMDNIISDLMDEGEGLDF

SEQ ID NO: 8 - protein, 10 aa, Artificial Sequence
  Synthetic Construct
GRKKRRQRRR

SEQ ID NO: 9 - protein, 12 aa, Artificial Sequence
  Synthetic Construct
GRKKRRQRRRPP

SEQ ID NO: 10 - protein, 27 aa, Artificial Sequence
  Synthetic Construct
GALFLGFLGAAGSTMGAWSQPKKKRKV

SEQ ID NO: 11 - protein, 21 aa, Artificial Sequence
  Synthetic Construct
KETWWETWWTEWSQPKKKRKV

SEQ ID NO: 12 - protein, 20 aa, Artificial Sequence
  Synthetic Construct
GLWRALWRLLRSLWRLLWRA

SEQ ID NO: 13 - protein, 8 aa, Artificial Sequence
  Synthetic Construct
RRRRRRRR

SEQ ID NO: 14 - protein, 28 aa, Artificial Sequence
  Synthetic Construct
GRKKRRQRRRPPSRRNAWGNQSYAELIS

SEQ ID NO: 15 - protein, 46 aa, Artificial Sequence
  Synthetic Construct
GRKKRRQRRRPPPRKGGSRRNAWGNQSYAELISQAIESAPEKRLTL

SEQ ID NO: 16 - protein, 33 aa, Artificial Sequence
  Synthetic Construct
GRKKRRQRRRPPLECDMDNIISDLMDEGEGLDF

SEQ ID NO: 17 - protein, 45 aa, Artificial Sequence
  Synthetic Construct
GRKKRRQRRRPPPQDLDLDMYMENLECDMDNIISDLMDEGEGLDF

SEQ ID NO: 18 - protein, 4 aa, Artificial Sequence
  Synthetic Construct
AWGN

SEQ ID NO: 19 - protein, 5 aa, Artificial Sequence
  Synthetic Construct
NAWGN

SEQ ID NO: 20 - protein, 6 aa, Artificial Sequence
  Synthetic Construct
RNAWGN

SEQ ID NO: 21 - protein, 7 aa, Artificial Sequence
  Synthetic Construct
RRNAWGN

SEQ ID NO: 22 - protein, 8 aa, Artificial Sequence
  Synthetic Construct
RRNAWGNQ

SEQ ID NO: 23 - protein, 10 aa, Artificial Sequence
  Synthetic Construct
SRRNAWGNQS

SEQ ID NO: 24 - protein, 10 aa, Artificial Sequence
  Synthetic Construct
RRNAWGNQSY

SEQ ID NO: 25 - protein, 13 aa, Artificial Sequence
  Synthetic Construct
SRRNAWGNQSYAE

SEQ ID NO: 26 - protein, 14 aa, Artificial Sequence
  Synthetic Construct
SRRNAWGNQSYAEL

SEQ ID NO: 27 - protein, 15 aa, Artificial Sequence
  Synthetic Construct
SRRNAWGNQSYAELI

SEQ ID NO: 28 - protein, 4 aa, Artificial Sequence
  Synthetic Construct
DLMD

SEQ ID NO: 29 - protein, 6 aa, Artificial Sequence
  Synthetic Construct
SDLMDE

SEQ ID NO: 30 - protein, 6 aa, Artificial Sequence
  Synthetic Construct
DNIISD

SEQ ID NO: 31 - protein, 10 aa, Artificial Sequence
  Synthetic Construct
IISDLMDEGE

SEQ ID NO: 32 - protein, 10 aa, Artificial Sequence
  Synthetic Construct
DNIISDLMDE

SEQ ID NO: 33 - protein, 14 aa, Artificial Sequence
  Synthetic Construct
DMDNIISDLMDEGE

SEQ ID NO: 34 - protein, 4 aa, Artificial Sequence
  Synthetic Construct
KKRR

SEQ ID NO: 35 - protein, 5 aa, Artificial Sequence
  Synthetic Construct
RKKRR

SEQ ID NO: 36 - protein, 6 aa, Artificial Sequence
  Synthetic Construct
RKKRRQ

SEQ ID NO: 37 - protein, 8 aa, Artificial Sequence
  Synthetic Construct
RKKRRQRR

SEQ ID NO: 38 - DNA, 21 bp, Artificial Sequence
  Synthetic Construct
ccagcttcagtcagcagttat

SEQ ID NO: 39 - DNA, 21 bp, Artificial Sequence
  Synthetic Construct
cgtccacgaagcagttcaaat

SEQ ID NO: 40 - DNA, 21 bp, Artificial Sequence
  Synthetic Construct
cgagtcagtatccagcccaat

SEQ ID NO: 41 - DNA, 21 bp, Artificial Sequence
  Synthetic Construct
cgggacaaagacaactaggtt

SEQ ID NO: 42 - DNA, 21 bp, Artificial Sequence
  Synthetic Construct
caagttcatcaaggttcacaa

SEQ ID NO: 43 - DNA, 20 bp, Artificial Sequence
  Synthetic Construct
aagggtgacagcaacagctc

SEQ ID NO: 44 - DNA, 21 bp, Artificial Sequence
  Synthetic Construct
ttctgcacacgaatgaacttg

SEQ ID NO: 45 - DNA, 24 bp, Artificial Sequence
  Synthetic Construct
gggttgtttcaatctaacagtcaa

SEQ ID NO: 46 - DNA, 22 bp, Artificial Sequence
  Synthetic Construct
caacattacggattgtgtagcc

SEQ ID NO: 47 - DNA, 20 bp, Artificial Sequence
  Synthetic Construct
acgagtggatggtccgtact

SEQ ID NO: 48 - DNA, 19 bp, Artificial Sequence
  Synthetic Construct
gtggcggatcgagttcttc

SEQ ID NO: 49 - DNA, 18 bp, Artificial Sequence
  Synthetic Construct
cttcgtctccgccatcag

SEQ ID NO: 50 - DNA, 18 bp, Artificial Sequence
  Synthetic Construct
ttgccaatctggacacca

SEQ ID NO: 51 - protein, 101 aa, Artificial Sequence
  Synthetic Construct
SSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRA

SEQ ID NO: 52 - protein, 101 aa, Artificial Sequence
  Synthetic Construct
GSRRNAWGNQSYAELISQAIESAPEKRLTLAQIYEWMVRTVPYFKDKGDSNSSAGWKNSIRHNLSLHSKFIKVHNEATGKSSWWMLNPEGGKSGKAPRRRA

SEQ ID NO: 53 - protein, 104 aa, Artificial Sequence
  Synthetic Construct
GYRRPLAHAKPPYSYISLITMAIQQAPGKMLTLSEIYQWIMDLFPYYRENQQRWQNSIRHSLSFNDCFVKVARSPDKPGKGSYWALHPSSGNMFENGCYLRRQK

SEQ ID NO: 54 - protein, 100 aa, Artificial Sequence
  Synthetic Construct
NYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPT

SEQ ID NO: 55 - protein, 41 aa, Artificial Sequence
  Synthetic Construct
DLDLDMFNGSLECDMESIIRSELMDADGLDFNFDSLISTQN

SEQ ID NO: 56 - protein, 40 aa, Artificial Sequence
  Synthetic Construct
DLDGMFIERLDCDMESIIRNDLMDGDTLDFNFDNVLPNQS

SEQ ID NO: 57 - protein, 37 aa, Artificial Sequence
  Synthetic Construct
DLDLDMYMENLECDMDNIISDLMDEGEGLDFNFEPDP

(19) United States
(12) Patent Application Publication
(10) Pub. No.: US 2013/0288980 A1
(43) Pub. Date: Oct. 31, 2013

(54) TARGETING SENESCENT AND CANCER CELLS FOR SELECTIVE KILLING BY INTERFERENCE WITH FOXO4

(71) Applicant: Buck Institute for Research on Aging, Novato, CA (US)

(72) Inventors: Peterus Leonardus-Josephus De Keizer, Utrecht (NL); Judith Campisi, Berkeley, CA (US); Remi-Martin Laberge, Novato, CA (US)

(21) Appl. No.: 13/830,790

(22) Filed: Mar. 14, 2013

Related U.S. Application Data
(60) Provisional application No. 61/619,322, filed on Apr. 2, 2012.

(51) Int. Cl. C07K 14/47; C07K 7/08

(52) U.S. Cl. CPC C07K 14/47; C07K 7/08
USPC 514/19.3; 435/375; 530/326; 530/324

(57) ABSTRACT

The present invention relates to agents that inhibit FOXO4 function and uses thereof in treating cancer and/or removing senescent cells in an individual.

### CROSS-REFERENCE TO RELATED APPLICATIONS

This application claims priority from U.S. Provisional Patent Application No. 61/619,322, filed Apr. 2, 2012, the contents of which are incorporated herein by reference in its entirety.

### FEDERAL FUNDING

This invention was made with Government support under Grant Nos. R37-AG09909 and P01-AG17242, awarded by the National Institutes of Health. The Government has certain rights in the invention.

### TECHNICAL FIELD

The present invention relates to compositions for inhibition of FOXO4 and uses thereof for treating cancer and/or removing senescent cells.

### BACKGROUND

DNA damage can lead to unrepaired or misrepaired (mutagenic) lesions that can in turn promote aging phenotypes and age-related pathology, including cancer (Vijg, J. et al. Nature 454, 1065-1071 (2008)). The development of cancer from damaged cells in vivo is restricted by two potent tumor suppressive mechanisms: apoptosis (programmed cell death) and cellular senescence (permanent arrest of cell proliferation). Apoptosis produces cross-linked cell fragments, which are cleared from the organism by engulfing macrophages and other cells. Non-dividing senescent cells can also be cleared by immune cells, but this process is inefficient. Consequently, senescent cells can remain in tissues. They have been shown to accumulate with age, and at sites of age-related pathology. Further, senescent cells can acquire secondary mutations that allow them to re-enter a proliferative state. Benign senescent lesions thus retain the capacity to become malignant.

In addition to the cell-autonomous growth arrest, senescent cells also alter the tissue microenvironment and possibly the systemic milieu by secreting pro-inflammatory cytokines, growth factors and matrix degrading proteases, a phenotype termed the senescence-associated secretory phenotype (SASP) (Coppe, J. P. et al. PLoS.Biol. 6, 2853-2868 (2008)). The SASP can accelerate aging phenotypes and cancer progression in a paracrine fashion. Indeed, it was recently shown in a mouse model of progeria that the killing of senescent cells by a suicide gene driven by a senescence-sensitive promoter could reverse certain aging phenotypes in vivo (Baker, D. J. et al. Nature 479, 232-236, (2011)). Thus, removal of senescent cells and senescence-escaped cancer cells by forcing them to undergo apoptosis has tremendous therapeutic potential for treating age-related pathologies, including cancer.

The present invention relates to agents that inhibit FOXO4 function and uses thereof in treating cancer and/or removing senescent cells.

All references cited herein, including patent applications and publications, are incorporated by reference in their entirety.

### SUMMARY OF THE INVENTION

Provided herein are methods of treating cancer in an individual comprising administering to the individual an effective amount of an agent that inhibits FOXO4 (e.g., human FOXO4), wherein the agent is used as an adjuvant therapy. Also provided herein are methods of conferring sensitivity to chemotherapy in cancer cells (e.g., in an individual such as human) comprising contacting the cancer cells with an effective amount of an agent that inhibits FOXO4. Also provided herein are methods of treating cancer in an individual comprising administering to the individual an effective amount of (a) an agent that inhibits FOXO4; and (b) at least one other chemotherapeutic agent. Also provided herein are methods of treating cancer (e.g., non-melanoma cancer) in an individual comprising administering to the individual an effective amount of an agent that inhibits FOXO4.

In some embodiments, the agent that inhibits FOXO4 is a peptide that inhibits FOXO4 function in a cell, wherein the peptide comprises an amino acid sequence that has at least 80% identity to a fragment of the FOXO4 (e.g., a fragment of SEQ ID NO:1). In some embodiments, the agent is a peptide comprising an amino acid sequence that has at least 80% identity to a fragment in the DNA binding domain of FOXO4 (e.g., a fragment of SEQ ID NO:2). In some embodiments, the agent is a peptide comprising an amino acid sequence that has at least 80% identity to a fragment in the C-terminal region of FOXO4 (e.g., a fragment of SEQ ID NO:3). In some embodiments, the agent is an antisense oligonucleotide targeting FOXO4. In some embodiments, the agent that inhibits FOXO4 function is through the use of hammerhead ribozyme, non-integrating or integrating lentivirus, micro RNA, or hairpin-based RNA interference (such as the hairpin RNAs used in the Examples).

In some embodiments, the agent is a peptide comprising an amino acid sequence that has at least 80% identity to a fragment in the DNA binding domain of FOXO4 (e.g., a fragment of SEQ ID NO:2). In some embodiments, the method further comprises administration of a second peptide, wherein the second peptide is a peptide comprising an amino acid sequence that has at least 80% identity to a fragment in the C-terminal region of FOXO4 (e.g., a fragment of SEQ ID NO:3).

Also provided herein are methods of treating cancer in an individual comprising administering to the individual an effective amount of (a) a peptide comprising an amino acid sequence that has at least 80% identity to a fragment in the DNA binding domain of FOXO4 (e.g., a fragment of SEQ ID NO:2); and/or (b) a peptide comprising an amino acid sequence that has at least 80% identity to a fragment in the C-terminal region of FOXO4 (e.g., a fragment of SEQ ID NO:3), wherein the peptide of (a) and/or peptide of (b) are used as an adjuvant therapy. Also provided here are methods of conferring sensitivity to chemotherapy in cancer cells (e.g., in an individual such as human) comprises contacting the cancer cells with an effective amount of (a) a peptide comprising an amino acid sequence that has at least 80% identity to a fragment in the DNA binding domain of FOXO4 (e.g., a fragment of SEQ ID NO:2); and/or (b) a peptide comprising an amino acid sequence that has at least 80% identity to a fragment in the C-terminal region of FOXO4 (e.g., a fragment of SEQ ID NO:3). Also provided herein are methods of treating cancer in an individual comprising administering to the individual an effective amount of (a) a peptide comprising an amino acid sequence that has at least 80% identity to a fragment in the DNA binding domain of FOXO4 (e.g., a fragment of SEQ ID NO:2); (b) a peptide comprising an amino acid sequence that has at least 80% identity to a fragment in the C-terminal region of FOXO4 (e.g., a fragment of SEQ ID NO:3); and/or (c) at least one other chemotherapeutic agent. Also provided herein are methods of treating cancer in an individual comprising administering to the individual an effective amount of (a) a peptide comprising an amino acid sequence that has at least 80% identity to a fragment in the DNA binding domain of FOXO4 (e.g., a fragment of SEQ ID NO:2); and/or (b) a peptide comprising an amino acid sequence that has at least 80% identity to a fragment in the C-terminal region of FOXO4 (e.g., a fragment of SEQ ID NO:3), wherein the cancer is not melanoma.

Also provided herein are peptides (e.g., isolated peptides) that inhibit FOXO4 function (e.g., inhibit FOXO4 function in a cell), wherein the peptide comprises an amino acid sequence that has at least about 80% identity (e.g., at least about 85%, 90%, 95%, 98%, 99%, or 100% identity) to a fragment of the FOXO4 (e.g., a fragment of SEQ ID NO:1). In some embodiments, the FOXO4 is human FOXO4. In some embodiments, the peptide is a fragment in SEQ ID NO:1. In some embodiments, the peptide is a fragment in the DNA binding domain (e.g., SEQ ID NO:2). In some embodiments, the peptide comprises WG. In some embodiments, the peptide is a fragment in the C-terminal region of FOXO4 (SEQ ID NO:3). In some embodiments, the peptide is a fragment in SEQ ID NO:1 and has at least about 5 amino acids (e.g., at least about any of 6, 7, 8, 9, 10, 11, 12, 13, 14, or 15 amino acids). In some embodiments, the peptide is a fragment in SEQ ID NO:1 and has about 5 amino acids (e.g., about any of 6, 7, 8, 9, 10, 11, 12, 13, 14, 15, 16, 17, 18, 19, or 20 amino acids).

In some embodiments, the peptide comprises amino acid sequence that has at least about 80% identity (e.g., at least about 85%, 90%, 95%, 98%, 99%, or 100% identity) to the sequence selected from the group consisting of (i) SRRNAWGNQSYAELIS, (ii) PRKGGSRRNAWGNQSYAELISQAIESAPEKRLTL; (iii) LECDMDNIISDLMDEGEGLDF; and (iv) PQDLDLDMYMENLECDMDNIISDLMDEGEGLDF. In some embodiments, the peptide comprises the amino acid sequence selected from the group consisting of (i) SRRNAWGNQSYAELIS, (ii) PRKGGSRRNAWGNQSYAELISQAIESAPEKRLTL; (iii) LECDMDNIISDLMDEGEGLDF; and (iv) PQDLDLDMYMENLECDMDNIISDLMDEGEGLDF.

In some embodiments, the peptide further comprises an amino acid sequence (e.g., at the N- or C-terminus) that facilitates entry into a cell. In some embodiments, the peptide further comprises at the N-terminus an amino acid sequence that facilitates entry of the peptide into a cell. In some embodiments, the sequence that facilitates entry into a cell comprises the amino acid sequence GRKKRRQRRR or GRKKRRQRRRPP. In some embodiments, the sequence that facilitates entry into a cell comprises a sequence such as GALFLGFLGAAGSTMGAWSQPKKKRKV, KETWWETWWTEWSQPKKKRKV, Ac-GLWRALWRLLRSLWRLLWRA-Cya, or octa-arginine (R(8)).

In some embodiments, the peptide comprises the sequence selected from the group consisting of (i) GRKKRRQRRRPPSRRNAWGNQSYAELIS; (ii) GRKKRRQRRRPPPRKGGSRRNAWGNQSYAELISQAIESAPEKRLTL; (iii) GRKKRRQRRRPPLECDMDNIISDLMDEGEGLDF; and (iv) GRKKRRQRRRPPPQDLDLDMYMENLECDMDNIISDLMDEGEGLDF.

In some embodiments, the peptide has a solubility (e.g., solubility in aqueous solution such as water) of at least about 1 mg/ml (at least about any of 2, 3, 4, 5, 6, 7, 8, 9, 10, 15, 20, 25, 30, 35, 40, 45, 50, 75, or 100 mg/ml).

In some embodiments, at least one peptide (e.g., 2, 3, 4, 5, 6, or 7 peptides) is used. For example, (a) a peptide comprising an amino acid sequence that has at least 80% identity to a fragment in the DNA binding domain of FOXO4 (e.g., a fragment in SEQ ID NO:2) and (b) a peptide comprising an amino acid sequence that has at least 80% identity to a fragment in the C-terminal region of FOXO4 (e.g., a fragment in SEQ ID NO:3) are used together to achieve an effect. At least one peptide provided herein, alone or in combination with another peptide, may inhibit the binding between FOXO4 and p53. In some embodiments, the at least one peptide, alone or in combination with another peptide, inhibits the binding between FOXO4 and p53, wherein the p53 is phosphorylated at serine 46. In some embodiments, the at least one peptide, alone or in combination with another peptide, induces apoptosis or killing of a cell (such as a cancer cell or a senescent cell) when the cell is brought in contact with the peptide.

Also provided are compositions comprising any one of the peptides provided herein. In some embodiments, (a) a peptide comprising an amino acid sequence that has at least 80% identity to a fragment in the DNA binding domain of FOXO4 (e.g., a fragment in SEQ ID NO:2) is used in combination with (b) a peptide comprising an amino acid sequence that has at least 80% identity to a fragment in the C-terminal region of FOXO4 (e.g., a fragment in SEQ ID NO:3). In some embodiments, there is provided a composition comprising (a) a peptide comprising an amino acid sequence that has at least 80% identity to a fragment in the DNA binding domain of FOXO4 (e.g., a fragment in SEQ ID NO:2); and/or (b) a peptide comprising an amino acid sequence that has at least 80% identity to a fragment in the C-terminal region of FOXO4 (e.g., a fragment in SEQ ID NO:3). In some embodiments, the composition comprises (a) a peptide comprising an amino acid sequence that has at least 80% identity to the sequence selected from the group consisting of (i) SRRNAWGNQSYAELIS, and (ii) PRKGGSRRNAWGNQSYAELISQAIESAPEKRLTL; and (b) a peptide comprising an amino acid sequence that has at least 80% identity to the sequence selected from the group consisting of (i) LECDMDNIISDLMDEGEGLDF; and (ii) PQDLDLDMYMENLECDMDNIISDLMDEGEGLDF. In some embodiments, the composition comprises (a) a peptide comprising an amino acid sequence selected from the group consisting of (i) SRRNAWGNQSYAELIS, and (ii) PRKGGSRRNAWGNQSYAELISQAIESAPEKRLTL; and (b) a peptide comprising an amino acid sequence selected from the group consisting of (i) LECDMDNIISDLMDEGEGLDF; and (ii) PQDLDLDMYMENLECDMDNIISDLMDEGEGLDF. In some embodiments, the composition further comprises a pharmaceutically acceptable carrier.

In some embodiments, there is provided a composition comprises (a) a peptide comprising an amino acid sequence PRKGGSRRNAWGNQSYAELISQAIESAPEKRLTL; and (b) a peptide comprising an amino acid sequence LECDMDNIISDLMDEGEGLDF. In some embodiments, there is provided a composition comprises (a) a peptide comprising an amino acid sequence GRKKRRQRRRPPPRKGGSRRNAWGNQSYAELISQAIESAPEKRLTL; and (b) a peptide comprising an amino acid sequence GRKKRRQRRRPPLECDMDNIISDLMDEGEGLDF. In some embodiments, the composition further comprises a pharmaceutically acceptable carrier.

In some embodiments, a peptide provided herein inhibits FOXO4 function (e.g., any of the FOXO4 function described herein). In some embodiments, the inhibition of FOXO4 function is achieved by at least two of the peptides provided herein, such as (a) a peptide comprising an amino acid sequence that has at least 80% identity to a fragment in the DNA binding domain of FOXO4 (e.g., a fragment in SEQ ID NO:2); and (b) a peptide comprising an amino acid sequence that has at least 80% identity to a fragment in the C-terminal region of FOXO4 (e.g., a fragment in SEQ ID NO:3).

Also provided herein are nucleic acids (e.g., isolated nucleic acids) encoding any one the peptides provided herein. Also provided herein are vectors (e.g., expression vectors) comprising any one of the nucleic acids provided herein. Also provided herein are cells comprising any one of the vectors or nucleic acids provided herein.

Also provided herein are methods of treating cancer in an individual comprising administering to the individual an effective amount of any one (or at least one) of the peptides or compositions provided herein. Also provided herein are methods of treating cancer in an individual comprising administering to the individual an effective amount of any one (or at least one) of the peptides or compositions provided herein, wherein the peptide or composition is used as adjuvant therapy. Also provided herein are methods of conferring sensitivity to radiation in cancer cells (e.g., in an individual such as human) comprises (e.g., in an individual such as human) contacting the cancer cells with an effective amount of any one (or at least one) of the peptides or compositions provided herein. Also provided herein are methods of treating cancer in an individual comprising administering to the individual an effective amount of any one (or at least one) of the peptides or compositions provided herein, wherein the peptide or composition is used in combination with radiation therapy. Also provided herein are methods of conferring sensitivity to chemotherapy (or chemotherapeutic agent) in cancer cells (e.g., in an individual such as human) comprises contacting the cancer cells with an effective amount of any one (or at least one) of the peptides or compositions provided herein. Also provided herein are methods of treating cancer in an individual comprising administering to the individual an effective amount of any one (or at least one) of the peptides or compositions provided herein, wherein the peptide or composition is used in combination with at least one other chemotherapeutic agent.

In some embodiments, there is provided a method of inducing apoptosis of cells (or removing or killing cells such as senescent cells or cancer cells, or inducing apoptosis of senescent cells or cancer cells) (e.g., in an individual such as human) comprising contacting the cells with an effective amount of any one (or at least one) of the peptides or compositions provided herein. Also provided are methods of treating an age-related disease or pathology or a symptom thereof (or reducing or alleviating one or more symptoms of an age-related disease) comprising administering an effective amount of any one (or at least one) of the peptides or compositions provided herein. Also provided are methods of treating a disease associated with senescent cells (e.g., accumulation of senescent cells) or pathology or a symptom thereof (or reducing or alleviating one or more symptoms of a disease associated with senescent cells) (e.g., in an individual such as human) comprising administering an effective amount of any one (or at least one) of the peptides or compositions provided herein. In some embodiments, the age-related disease or senescent cell-related disease and/or their pathology may be any one or more of Alzheimer's disease, Huntington's disease, diseases associated with cataracts, atherosclerosis, chronic obstructive pulmonary disease (COPD), emphysema, diabetic ulcer, kyphosis, herniated intervertebral discs, osteoarthritis, osteoporosis, Parkinson's disease, renal disease, renal failure, or sarcopenia.

In some embodiments, there is provided a method of treating cancer in an individual comprising administering to the individual an effective amount of (a) a peptide comprising an amino acid sequence that has at least 80% identity to a fragment in the DNA binding domain of FOXO4 (e.g., a fragment in SEQ ID NO:2); and/or (b) a peptide comprising an amino acid sequence that has at least 80% identity to a fragment in the C-terminal region of FOXO4 (e.g., a fragment in SEQ ID NO:3). Also provided herein are methods of conferring sensitivity to radiation in cancer cells (e.g., in an individual such as human) comprises contacting the cancer cells with an effective amount of (a) a peptide comprising an amino acid sequence that has at least 80% identity to a fragment in the DNA binding domain of FOXO4 (e.g., a fragment in SEQ ID NO:2); and/or (b) a peptide comprising an amino acid sequence that has at least 80% identity to a fragment in the C-terminal region of FOXO4 (e.g., a fragment in SEQ ID NO:3). Also provided herein are methods of treating cancer in an individual comprising administering to the individual an effective amount of (a) a peptide comprising an amino acid sequence that has at least 80% identity to a fragment in the DNA binding domain of FOXO4 (e.g., a fragment in SEQ ID NO:2); and/or (b) a peptide comprising an amino acid sequence that has at least 80% identity to a fragment in the C-terminal region of FOXO4 (e.g., a fragment in SEQ ID NO:3), wherein the peptide(s) are used in combination with radiation therapy. Also provided herein are methods of inducing apoptosis of cells (or killing or removing cells such as senescent cells or cancer cells, or inducing apoptosis of senescent cells or cancer cells) (e.g., in an individual such as human) comprising contacting the cells with an effective amount of (a) a peptide comprising an amino acid sequence that has at least 80% identity to a fragment in the DNA binding domain of FOXO4 (e.g., a fragment in SEQ ID NO:2); and/or (b) a peptide comprising an amino acid sequence that has at least 80% identity to a fragment in the C-terminal region of FOXO4 (e.g., a fragment in SEQ ID NO:3).

In some embodiments of any of the methods provided herein, the method further comprises radiation therapy (such as ionizing radiation or X-ray). In some embodiments of any of the methods provided herein, the method further comprises administration of at least one other chemotherapeutic agent. In certain embodiments, at least one other chemotherapeutic agent is a therapeutic antibody, a topoisomerase inhibitor, an antimetabolite, a platinum-based agent, an alkylating agent, a tyrosine kinase inhibitor, an Anthracycline antibiotic, an anti-angiogenic agent, or a vinca alkaloid. In some embodiments, the at least one other chemotherapeutic agent is a RAF inhibitor (e.g., RAF265), BRAF^{V600E} inhibitor (e.g., PLX4032 or vemurafenib), or MEK inhibitor (e.g., AZD6244 or selumetinib). In some embodiments, the at least one other chemotherapeutic agent is 5-FU (or fluorouracil), cisplatin, dacarbazine, RAF265, PLX4032, AZD6244 (selumetinib), gemcitabine, capecitabine, methotrexate (anti-folic acid), vinblastine, doxorubicin, or mitoxantrone.

In some embodiments, the cancer is melanoma. In some embodiments, the cancer is not melanoma. In some embodiments, the cancer is skin cancer (such as melanoma), mammary cancer, breast cancer, prostate cancer, pancreatic cancer, ovarian cancer, glioblastoma, renal cancer, or bladder cancer. In some embodiments, the cancer does not comprise mutation in p53. (e.g., the cancer is wildtype for p53).

In some embodiments, any of the agents that inhibit FOXO4, the peptides or compositions provided herein has one or more of the following uses: (1) use in treating cancer such as use as an adjuvant therapy; (2) conferring sensitivity to radiation or chemotherapy in cells (e.g., cancer cells); (3) use in combination with radiation therapy (e.g., ionizing radiation or X-ray) or surgery; (4) use in combination with at least one other chemotherapeutic agent; (5) use in treating an age-related disease or pathology or a symptom thereof (e.g., reducing or alleviating a symptom thereof); (6) use in reducing, alleviating, or delaying a symptom associated with an ageing phenotype or senescence-associated secretory phenotype (SASP), or use in delaying development of SASP; (7) use in delaying or preventing development of an age-related disease or pathology or a symptom thereof; (8) use in reducing inflammation after radiation therapy or administration of a chemotherapeutic agent; (9) use in reducing recurrence of cancer; (10) use in reducing metastasis of tumor or cancer. In some embodiments, any of the agents that inhibit FOXO4, the peptides or compositions provided herein, and at least one other chemotherapeutic agent are administered concurrently or sequentially. In some embodiments, any of the agents that inhibit FOXO4, the peptides or compositions provided herein is administered before or after radiation therapy or administration of at least one other chemotherapeutic agent.

In some embodiments, any of the agents that inhibit FOXO4 (e.g., hairpin-based interference RNA) or the peptides provided herein may be delivered by liposomes or cell permeable peptide (“CPP”). In some embodiments, the CPP is fused to a peptide provided herein. The CPP may be any of the following: MPG (GALFLGFLGAAGSTMGAWSQPKKKRKV), Pep-1 (KETWWETWWTEWSQPKKKRKV), CADY (Ac-GLWRALWRLLRSLWRLLWRA-Cya) or octa-arginine (R(8)).

It is to be understood that one, some, or all of the properties of the various embodiments described herein may be combined to form other embodiments of the present invention. These and other aspects of the invention will become apparent to one of skill in the art.

### BRIEF DESCRIPTION OF THE FIGURES

FIG. 1. FOXO4 mRNA and protein levels increase in response to senescence-inducing DNA damage. A) FOXO4 and FOXO3 protein expression in normal human HCA2 fibroblasts after treatment with 10Gy XRAY. B) FOXO4 mRNA expression determined by quantitative real-time PCR in normal human IMR90 fibroblasts after treatment with 10Gy XRAY. C) Punctate localization of FOXO4 in DNA-damaged IMR90 fibroblasts positive for a marker of senescence—Senescence-associated-β-Galactosidase (SA-β-GAL). D) Localization of FOXO4 in PML bodies adjacent to DNA-SCARS (53BP1 and ATM substrate) and mutually exclusive with heterochromatin (DAPI) in DNA damaged, senescent IMR90. Immunofluorescence-mediated detection of FOXO4 and other senescence markers in XRAY-senescent IMR90 fibroblasts showing the following: Top left panel: FOXO4 foci are mutually exclusive with Senescence-Associated Heterochromatin Foci (SAHFs; stained by DAPI), a marker of inactive transcription. Top right panel: FOXO4 foci strongly co-localize with PML bodies. Bottom left: FOXO4 foci are partially adjacent to substrates of the DNA-damage activated kinase ATM, a marker of DNA-SCARS that are necessary for senescence-associated growth arrest and the senescence-associated secretory phenotype. Bottom right pane: FOXO4 foci are partially adjacent to 53BP1, another marker of DNA-SCARS. E) FOXO1, FOXO3a and FOXO4 mRNA expression determined by quantitative real-time PCR in normal human IMR90 fibroblasts after treatment with 10Gy XRAY.

FIG. 2. Knockdown of FOXO4 results in apoptosis rather than senescence in DNA-damaged IMR90 fibroblasts. A) Reduction in punctate FOXO4 staining and FOXO4 mRNA expression after 8 days 10Gy XRAY in HCA2 fibroblasts infected three distinct hairpins targeted at FOXO4. Note that shFOXO4-1 and -2 induce knockdown of FOXO4 mRNA and reduction in punctate FOXO4, whereas shFOXO4-3 does not. B) Increased cytoplasmic Cytochrome C staining (a marker of apoptosis; arrowheads) in DNA-damaged IMR90 cells infected with knockdown against FOXO4. The percentage apoptotic cells is quantified. C) Increased TUNEL staining (a second marker of apoptosis) in similar conditions a B.

FIG. 3. DNA damage (XRAY) induces expression of Ser-46 phosphorylated p53 and interference with its kinase blocks apoptosis in FOXO4-depleted cells. A) Protein levels of Ser-46 phosphorylated and total p53 vs. FOXO4 in DNA-damaged IMR90 fibroblasts. B) The increase in cytoplasmic Cytochrome C (apoptosis; arrows) in DNA-damaged IMR90 cells deprived of FOXO4 is blocked upon knockdown of the kinase of Ser46-p53-HIPK2. The localization of Cytochrome C was determined and was found to be mitochondrial in panel 1,3 and 4 and diffuse in panel 2, indicating apoptosis. The top and bottom rows are similar, except that the bottom row also shows DAPI staining as a marker for nuclei.

FIG. 4. Senescence-inducing levels of irradiation induce apoptosis in the kidney cortex of foxo4^{-/-} mice. A) Visual example of TUNEL positivity as marker for apoptosis and total cell number indicated by DAPI and B) quantification of the percentage of TUNEL-positive cells of the cortex of wildtype mice and foxo4−/− mice 7 days after treatment with senescence-inducing levels of ionizing radiation (5Gy). Three mice were irradiated per group and the percentage TUNEL-positive cells was objectively determined by Cellprofiler software over three images per cortex.

FIG. 5. Senescence occurs with normal aging in wildtype mouse kidneys and at an accelerated pace in TTD premature aging mice. A) Kidneys from wildtype mice (wt) and accelerated aging trichothiodystrophy (TTD) mice of different ages were stained for the presence of senescent cells by Senescence-Associated β-Galactosidase (SA-β-GAL) staining. Senescence increases progressively in time in wildtype mice and at an accelerated pace in TTD mice, as indicated by mild, some and severe senescence, respectively.

FIG. 6. FOXO4 inhibition induces apoptosis in senescent TTD kidneys ex vivo. Kidneys from 13 week old wildtype (wt) and premature aging TTD mice, were sliced in 300 μM slices and cultured ex vivo. At 5 days post infection with either shGFP or shFOXO4-expressing lentiviral particles slices were processed for TUNEL staining as a marker for apoptosis. A) Visual presentation of apoptosis characterized by TUNEL positivity in TTD kidney slices where FOXO4 expression was reduced by lentivirus-based knockdown. B) Quantification of the percentage TUNEL-positive cells in images of wt and TTD kidney slices infected with control or FOXO4 knockdown lentiviral particles. Quantification was objectively performed using Cellprofiler software.

FIG. 7. FOXO4-p53 blocking peptides induce apoptosis in TTD, but not wt kidneys ex vivo. A) Kidneys from 13 week old wildtype (wt) and premature aging TTD mice, were sliced in 300 μM slices and cultured ex vivo. The slices were subsequently incubated with 12.5 μM FOXO4-p53 blocking peptide mix on day 0 and again in fresh media on day 2. On day 5 the slices were stained for TUNEL positivity, which was objectively quantified using Cellprofiler software.

FIG. 8. Model explaining how FOXO4 restrains apoptosis in favor of senescence in DNA-damaged cells. Left: DNA damage results in Ser46-phosphorylation of p53, which normally triggers an apoptosis response. FOXO4 levels however also rise and FOXO4 physically associates with p53 resulting in p21^{cip1}-mediated cell cycle arrest rather than apoptosis. Right: When FOXO4 expression is decreased or the interaction with p53 inhibited ser46-phosphotylated p53 now engages the apoptotic machinery.

FIG. 9. BRAF^{V600E}-induces the interaction between FOXO4 and p53 and FOXO4 depletion increases sensitivity of A375 cells to undergo apoptosis by BRAF^{V600E}-inhibition. A+B) Co-immunoprecipitation with endogenous p53 for ectopically expressed FOXO4 in absence or presence of BRAF^{V600E} co expression showing increased p53 FOXO4 interaction when BRAF^{V600E} is present (A) and its downstream signaling is uninhibited by the MEK inhibitor U0126 (B) (available at http://igitur-archive.library.uu.nl/dissertations/2009-0630-200608/UUindex.html; last accessed on Mar. 30, 2012). Phospho-JNK and phospho-ERK are shown as markers of active BRAF^{V600E} signaling. C) Increased sensitivity of BRAF^{V600E}-mutated metastatic A375 melanoma cells to the BRAF^{V600E}-specific inhibitor PLX4032 (Vemurafenib) upon depletion of FOXO4. A375 cells stably infected with control short hairpin or the two short hairpins that were shown do induce knockdown of FOXO4 in FIG. 2. were treated with three different concentrations 0.5 μM, 2 μM and 10 μM of PLX4032 and after 40 h the percentage cytochrome C released from the mitochondria (marker of apoptosis) was scored. The immunofluorescence panels show staining of Cytochrome C. The top, middle and bottom left panels all show mitochondrial Cytochrome C staining. The top right panel shows some a few cells in which this localization is lost, but importantly the number of cells with diffused Cytochrome C is significantly higher in the middle and bottom right panels, visualizing what is scored in the bar graph. D) Reduced viability in A375 melanoma cells treated for 6 days with 0.5 μM PLX4032 in the presence of 50 μM FOXO4-p53 blocking peptide mix.

FIG. 10. Spontaneous apoptosis in NRAS^{Q61K}-mutated D04 human melanoma cells. A) D04 melanoma cells were infected with control short hairpin (shGFP) or the three hairpins targeted against FOXO4 presented in FIG. 2, selected for 48 h with puromycin, refreshed for 24 h and replated. Their colony forming potential was determined after 10 days, and their cellular morphology and Cytochrome C release after 5 days. shFOXO4 infected D04 spontaneously apoptosed as determined by reduced colony formation, rounding up of the cells and release of Cytochrome C from the mitochondria. FIG. 10A) Bottom two rows show localization of Cytochrome C. They are identical, except that the bottom row also shows DAPI staining to indicate nuclei. Whereas in the far left and far right panels Cytochrome C is localized in its characteristic mitochondrial location, in the middle two panels there are a number of cells indicated by the arrowheads that show diffused Cytochrome C staining and are apoptotic. B) D04 cells were plated and incubated with 50 μM of the p53-FOXO4 blocking peptide mix. The number of cells was scored by Coulter Counter after 10 days.

FIG. 11. Sensitization of melanoma cells other types of cancer to additional types of chemotherapy. A) A375 melanoma cells were incubated with the FOXO4-p53 blocking peptide mix in the presence or absence of 2 μM Cisplatin and after 4 days cellular morphology was visualized. Note the increase in rounded-up (apoptotic) cells when Cisplatin was combined with the peptide mix. B) MCF7 mammary carcinoma cells were infected with the short hairpins against FOXO4 and after puromycin selection treated with 5 μM 5′-fluoro-uracil. 40 h later the percentage Cytochrome C-release was scored. The immunofluorescence panels show staining of Cytochrome C. The top, middle and bottom left panels all show mitochondrial Cytochrome C staining. The top right panel shows some a few cells in which this localization is lost, but importantly the number of cells with diffused Cytochrome C is significantly higher in the middle and bottom right panels, visualizing what is scored in the bar graph. C) B16F10Luc mouse melanoma cells were infected with a mouse-specific short hairpin against FOXO4 and short hairpin #2 against human FOXO4 from FIG. 2 which shares sequence homology with mouse FOXO4. After puromycin selection cells were incubated with 10 μM Cisplatin and after 7 days cellular viability was determined by CellTiter 96® AQueous One Solution Cell Proliferation Assay (MTS).

FIG. 12. FOXO4 inhibition on sensitizes A375 melanoma cells to inhibitors of the BRAF/MEK pathway. A) A375 cells stably infected with a control shRNA (shGFP) or an shRNA against FOXO4 (shFOXO4) were treated with the indicated concentrations of the BRAF^{V600E} inhibitor PLX4032 and the percentage of the surviving fraction on was determined by Aqueous CellQuant assay either after day 6 (left panel) or day 11 (right panel). Note that for the right panel the cells were refreshed at day 7 with the exact same concentration on of PLX4032. B) Similar experiment as in A), except with the general RAF inhibitor RAF265. C) Similar experiment as in A), except with the MEK inhibitor AZD6244.

FIG. 13. FOXO4 inhibition sensitizes A375 melanoma cells to Trametinib. A) A375 melanoma cells were lentivirally infected with control (shGFP) or FOXO4 knockdown (shFOXO4) constructs and selected for infected clones by antibiotic-treatment. Equal amounts were plated for colony assay. The next day the cells were exposed to different concentrations of Trametinib (20 nM and 80 nM, respectively) or Mock-treated. One week later the amount of colony formation was scored as percentage over Mock-treatment. shFOXO4-infected A375 cells showed a markedly increased sensitivity to Trametinib.

FIG. 14. A) Amino acid sequence alignment of DNA binding domains in FOXO3a, FOXO4, FOXA3, and FOXP2. B) Amino acid sequence alignment of C-terminal region in FOXO3a, FOXO1, and FOXO4.

### DETAILED DESCRIPTION

The invention described herein provides, inter alia, compositions of agents that inhibit FOXO4 function and methods for uses thereof in treating cancer and/or removing senescent cells in an individual.

### Definitions

As used herein, “treatment” or “treating” is an approach for obtaining beneficial or desired results including and preferably clinical results. For purposes of this invention, beneficial or desired clinical results include, but are not limited to, one or more of the following: reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

An “individual” or a “subject” is a mammal, more preferably a human. Mammals also include, but are not limited to, farm animals, sport animals, pets (such as cats, dogs, horses), primates, mice and rats.

As used herein, an “effective dosage” or “effective amount” of agent, peptide, drug, compound, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent and preferably stop) cancer cell infiltration into peripheral organs; inhibit (i.e., slow to some extent and preferably stop) tumor metastasis; inhibiting, to some extent, tumor growth; and/or relieving to some extent one or more of the symptoms associated with the disorder. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of an agent, peptide, drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

The term “isolated” as used herein may indicate that an agent, drug, or peptide that has been identified and separated and/or recovered from a component of its natural environment.

As used herein and in the appended claims, the singular forms “a,” “an,” and “the” include plural reference unless the context clearly indicates otherwise.

Reference to “about” a value or parameter herein includes (and describes) embodiments that are directed to that value or parameter per se. For example, description referring to “about X” includes description of “X.”

It is understood that aspect and variations of the invention described herein include “consisting” and/or “consisting essentially of” aspects and variations.

Agents that Inhibit FOXO4

The present disclosure provides agents that inhibit FOXO4 (e.g., human FOXO4) and uses thereof. An agent that inhibits FOXO4 as provided herein does not necessarily require that there is 100% of inhibition of FOXO4, e.g., an agent may inhibit FOXO4 to some extent. An agent that inhibits FOXO4 may refer to an agent that inhibits the expression of FOXO4, lowers the level of FOXO4 mRNA or protein level in a cell, inhibits the binding between FOXO4 and other proteins, affects the localization of FOXO4, regulates FOXO4 spatiotemporally, and/or affect the function (e.g., activity) of FOXO4. For example, an agent that inhibits FOXO4 may refer to an agent that inhibits the binding between FOXO4 and p53. Methods of measuring whether an agent inhibits FOXO4 may be by measuring expression of FOXO4, mRNA of FOXO4, binding of FOXO4 and other proteins such as p53, and/or ability to induce apoptosis of cells when brought in contact with such agent. In some embodiments, there is provided an agent that inhibits the function of human FOXO4. In some embodiments, at least two agents (e.g., 2, 3, 4, 5, 6, or 7) are used together to achieve an effect. In some embodiments, the agent that inhibits FOXO4 (e.g., a peptide) is isolated or purified.

Provided herein are agents that inhibit function, effect or activity of FOXO4, such as inhibit a function, effect or activity of FOXO4 as provided herein. In some embodiments, the agent inhibits the function, effect or activity of FOXO4 by at least about 5%, 10%, 15%, 20%, 25%, 30%, 35%, 40%, 45%, or 50% as compared to when the agent is not used (i.e., negative control). In other embodiments, the inhibition is at least about 55%, 60%, 65%, 70%, 75%, 80%, 85%, 90% or 95% of the function, effect or activity of FOXO4 as compared to when the agent is not used (i.e., negative control).

An agent that inhibits FOXO4 may be a small molecule, a peptide or a nucleotide such as an oligonucleotide (e.g., antisense oligonucleotide, microRNA, or hairpin-based interference RNA). In some embodiments, the agent is an antisense oligonucleotide targeting FOXO4. In some embodiments, an agent that inhibits FOXO4 function is through the use of hammerhead ribozyme, non-integrating or integrating lentivirus, micro RNA, or hairpin-based RNA interference (such as the hairpin RNAs used in the Examples).

In some embodiments, an agent that inhibits FOXO4 is a peptide that inhibits FOXO4 function in a cell. Such peptide may comprise an amino acid sequence that share certain identity or homology to a fragment of the FOXO4 (e.g., a fragment of SEQ ID NO:1). Such peptide may inhibit FOXO4 function by competing with FOXO4.

In some embodiments, an agent that inhibits FOXO4 comprises an amino acid sequence that has at least 80% identity to a fragment of the FOXO4 (e.g., a fragment of SEQ ID NO:1). For example, the peptide comprises an amino acid sequence that has about or at least about 80% identity (e.g., about or at least about 85%, 90%, 95%, 98%, 99%, or 100% identity) to a fragment of the FOXO4 (e.g., a fragment of SEQ ID NO:1). In some embodiments, the agent is a peptide comprising an amino acid sequence that has about or at least about 80% identity (e.g., about or at least about 85%, 90%, 95%, 98%, 99%, or 100% identity) to a fragment in the DNA binding domain of FOXO4 (e.g., a fragment of SEQ ID NO:2). In some embodiments, the agent is a peptide comprising an amino acid sequence that has about or at least about 80% identity (e.g., about or at least about 85%, 90%, 95%, 98%, 99%, or 100% identity) to a fragment in the C-terminal region of FOXO4 (e.g., a fragment of SEQ ID NO:3). In some embodiments, the FOXO4 is human FOXO4.

In some embodiments, the agent is a peptide comprising an amino acid sequence that has about or at least about 80% identity (e.g., about or at least about 85%, 90%, 95%, 98%, 99%, or 100% identity) to a fragment in SEQ ID NO:2, wherein the fragment in SEQ ID NO:2 is one of the following: WG, AWGN, NAWGN, RNAWGN, RRNAWGN, RRNAWGNQ, SRRNAWGNQS, RRNAWGNQSY, SRRNAWGNQSYAE, SRRNAWGNQSYAEL, SRRNAWGNQSYAELI, or SRRNAWGNQSYAELIS. In some embodiments, the agent is a peptide comprising the amino acid sequence WG, AWGN, NAWGN, RNAWGN, RRNAWGN, RRNAWGNQ, SRRNAWGNQS, RRNAWGNQSY, SRRNAWGNQSYAE, SRRNAWGNQSYAEL, SRRNAWGNQSYAELI, or SRRNAWGNQSYAELIS. In some embodiments, the agent is a peptide comprising an amino acid sequence that has about or at least about 80% identity (e.g., about or at least about 85%, 90%, 95%, 98%, 99%, or 100% identity) to a fragment in SEQ ID NO:2, wherein the fragment in SEQ ID NO:2 is SRRNAWGNQSYAELIS or PRKGGSRRNAWGNQSYAELISQAIESAPEKRLTL.

In some embodiments, the agent is a peptide comprising an amino acid sequence that has about or at least about 80% identity (e.g., about or at least about 85%, 90%, 95%, 98%, 99%, or 100% identity) to a fragment in SEQ ID NO:3, wherein the fragment in SEQ ID NO:3 is one of the following: DLMD, SDLMDE, DNIISD, IISDLMDEGE, DNIISDLMDE, DMDNIISDLMDEGE, or LECDMDNIISDLMDEGEGLDF. In some embodiments, the agent is a peptide comprising the amino acid sequence DLMD, SDLMDE, DNIISD, IISDLMDEGE, DNIISDLMDE, DMDNIISDLMDEGE, or LECDMDNIISDLMDEGEGLDF. In some embodiments, the agent is a peptide comprising an amino acid sequence that has about or at least about 80% identity (e.g., about or at least about 85%, 90%, 95%, 98%, 99%, or 100% identity) to a fragment in SEQ ID NO:3, wherein the fragment in SEQ ID NO:3 is LECDMDNIISDLMDEGEGLDF or PQDLDLDMYMENLECDMDNIISDLMDEGEGLDF.

In some embodiments, the peptide comprises a fragment in SEQ ID NO:1, 2, or 3 that has at least about 5 amino acids (e.g., at least about any of 6, 7, 8, 9, 10, 11, 12, 13, 14, or 15 amino acids). In some embodiments, the peptide comprises a fragment in SEQ ID NO:1, 2, or 3 that has about 5 amino acids (e.g., about any of 6, 7, 8, 9, 10, 11, 12, 13, 14, 15, 16, 17, 18, 19, or 20 amino acids).

In some embodiments, the peptide comprises amino acid sequence that has at least about 80% identity (e.g., at least about 85%, 90%, 95%, 98%, 99%, or 100% identity) to the sequence selected from the group consisting of (i) SRRNAWGNQSYAELIS, (ii) PRKGGSRRNAWGNQSYAELISQAIESAPEKRLTL; (iii) LECDMDNIISDLMDEGEGLDF; and (iv) PQDLDLDMYMENLECDMDNIISDLMDEGEGLDF. In some embodiments, the peptide comprises the amino acid sequence selected from the group consisting of (i) SRRNAWGNQSYAELIS, (ii) PRKGGSRRNAWGNQSYAELISQAIESAPEKRLTL; (iii) LECDMDNIISDLMDEGEGLDF; and (iv) PQDLDLDMYMENLECDMDNIISDLMDEGEGLDF.

In some embodiments, the peptide further comprises an amino acid sequence (e.g., at the N- or C-terminus) that facilitates entry into a cell. In some embodiments, the peptide further comprises at the N-terminus an amino acid sequence that facilitates entry of the peptide into a cell (e.g., TAT sequence). In some embodiments, the sequence that facilitates entry into a cell comprises the amino acid sequence KKRR, RKKRR, RKKRRQ, RKKRRQRR, GRKKRRQRRR or GRKKRRQRRRPP. In some embodiments, the peptide further comprises a cell permeable peptide (“CPP”), such as primary amphipatic peptide MPG (GALFLGFLGAAGSTMGAWSQPKKKRKV), Pep-1 (KETWWETWWTEWSQPKKKRKV), secondary amphipathic peptide CADY (Ac-GLWRALWRLLRSLWRLLWRA-Cya) or octa-arginine (R(8)).

In some embodiments, the peptide comprises the sequence selected from the group consisting of (i) GRKKRRQRRRPPSRRNAWGNQSYAELIS; (ii) GRKKRRQRRRPPPRKGGSRRNAWGNQSYAELISQAIESAPEKRLTL; (iii) GRKKRRQRRRPPLECDMDNIISDLMDEGEGLDF; and (iv) GRKKRRQRRRPPPQDLDLDMYMENLECDMDNIISDLMDEGEGLDF.

In any of the embodiments herein, the peptides are less than about 500, 400, 300, 200, or 100 amino acids in length. In any of the embodiments herein, the peptides are less than about 90, 85, 80, 75, 70, 65, 60, 55, 50, 45, 40, 35, 30, 25, 20, or 15 amino acids in length. In any of the embodiments herein, the peptides are greater than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acid in length. In any of embodiments herein, the peptide has a length that is any combination of the upper limits of length and lower limits of length as recited above.

In some embodiments, the peptide has a solubility (e.g., solubility in aqueous solution such as water) of at least about 1 mg/ml (at least about any of 2, 3, 4, 5, 6, 7, 8, 9, 10, 15, 20, 25, 30, 35, 40, 45, 50, 75, or 100 mg/ml).

In some embodiments, an agent that inhibits FOXO4 may be delivered by using cell permeable peptides (such as, but not limited to, CADY, MPG and Pepl) or by using octa-arginine.

In some embodiments, an agent that inhibits FOXO4 inhibits the binding between FOXO4 and p53. In some embodiments, the agent inhibits the binding between FOXO4 and p53, wherein the p53 is phosphorylated at serine 46. In some embodiments, the peptide induces apoptosis of a cell (such as a cancer cell or a senescent cell) when the cell is treated with the peptide.

Also provided are compositions comprising at least one (e.g., 2, 3, 4, or 5) of the agents or peptides provided herein. These agents or peptides, when used together, inhibit function of FOXO4 such as inhibit binding between FOXO4 and p53. In some embodiments, (a) a peptide comprising an amino acid sequence that has at least 80% identity to a fragment in the DNA binding domain of FOXO4 (e.g., a fragment in SEQ ID NO:2) is used in combination with (b) a peptide comprising an amino acid sequence that has at least 80% identity to a fragment in the C-terminal region of FOXO4 (e.g., a fragment in SEQ ID NO:3). In some embodiments, there is provided a composition comprising (a) a peptide comprising an amino acid sequence that has at least 80% identity to a fragment in the DNA binding domain of FOXO4 (e.g., a fragment in SEQ ID NO:2); and/or (b) a peptide comprising an amino acid sequence that has at least 80% identity to a fragment in the C-terminal region of FOXO4 (e.g., a fragment in SEQ ID NO:3). In some embodiments, the composition comprises (a) a peptide comprising an amino acid sequence that has at least 80% identity to the sequence selected from the group consisting of (i) SRRNAWGNQSYAELIS, and (ii) PRKGGSRRNAWGNQSYAELISQAIESAPEKRLTL; and (b) a peptide comprising an amino acid sequence that has at least 80% identity to the sequence selected from the group consisting of (i) LECDMDNIISDLMDEGEGLDF; and (ii) PQDLDLDMYMENLECDMDNIISDLMDEGEGLDF. In some embodiments, the composition comprises (a) a peptide comprising an amino acid sequence selected from the group consisting of (i) SRRNAWGNQSYAELIS, and (ii) PRKGGSRRNAWGNQSYAELISQAIESAPEKRLTL; and (b) a peptide comprising an amino acid sequence selected from the group consisting of (i) LECDMDNIISDLMDEGEGLDF; and (ii) PQDLDLDMYMENLECDMDNIISDLMDEGEGLDF. In some embodiments, there is provided a composition comprises (a) a peptide comprising an amino acid sequence PRKGGSRRNAWGNQSYAELISQAIESAPEKRLTL; and (b) a peptide comprising an amino acid sequence LECDMDNIISDLMDEGEGLDF. In some embodiments, there is provided a composition comprises (a) a peptide comprising an amino acid sequence GRKKRRQRRRPPPRKGGSRRNAWGNQSYAELISQAIESAPEKRLTL; and (b) a peptide comprising an amino acid sequence GRKKRRQRRRPPLECDMDNIISDLMDEGEGLDF. In some embodiments, the composition further comprises a pharmaceutically acceptable carrier.

In some embodiments, any one of the peptides inhibits FOXO4 function (e.g., any of the FOXO4 function described herein). In some embodiments, the inhibition of FOXO4 function is achieved by two or at least two of the peptides provided herein, such as (a) a peptide comprising an amino acid sequence that has at least 80% identity to a fragment in the DNA binding domain of FOXO4 (e.g., a fragment in SEQ ID NO:2); and (b) a peptide comprising an amino acid sequence that has at least 80% identity to a fragment in the C-terminal region of FOXO4 (e.g., a fragment in SEQ ID NO:3). Thus, (a) a peptide comprising an amino acid sequence that has at least 80% identity to a fragment in the DNA binding domain of FOXO4 (e.g., a fragment in SEQ ID NO:2) may be used in combination with (b) a peptide comprising an amino acid sequence that has at least 80% identity to a fragment in the C-terminal region of FOXO4 (e.g., a fragment in SEQ ID NO:3).

Any of the agents that inhibit FOXO4 provided herein may have one or more of the following: inhibits the expression of FOXO4, lowers the level of FOXO4 mRNA or protein level in a cell, inhibits the binding between FOXO4 and other proteins such as p53, induces apoptosis of cells (e.g., cancer cells or senescent cells) when brought in contact with such agent, increases sensitivity of cells to radiation or chemotherapeutic agent(s), delay recurrence of cancer or tumor, delay recurrence of chemotherapeutic agent-resistant cancer or tumor, reduce tumor metastasis, reduce inflammation after chemotherapy or radiation therapy.

An agent that inhibits FOXO4 may show one or more of the following: inhibits the expression of FOXO4, lowers the level of FOXO4 mRNA or protein level in a cell, or inhibits the binding between FOXO4 and other proteins such as p53. Methods of measuring whether an agent inhibits FOXO4 may be by measuring expression of FOXO4, mRNA of FOXO4, binding of FOXO4 and other proteins such as p53, and/or ability to induce apoptosis of cells when brought in contact with such agent. Methods of measuring mRNA or protein level of FOXO4, binding of FOXO4 and other proteins, and apoptosis of cells are known in the field and may also be according to the methods provided in the Examples described herein.

In some embodiments, any of the agents that inhibit FOXO4, the peptides or compositions provided herein has one or more of the following uses: (1) use as an adjuvant therapy; (2) conferring sensitivity to radiation or chemotherapy in cells (e.g., cancer cells); (3) use in combination with radiation therapy (e.g., ionizing radiation or X-ray) or surgery; (4) use in combination with at least one other chemotherapeutic agent; (5) use in treating an age-related disease or symptom thereof (e.g., reducing or alleviating a symptom thereof); (6) use in reducing or alleviating a symptom associated with an ageing phenotype or SASP; (7) use in reducing inflammation after radiation therapy or administration of a chemotherapeutic agent; (8) use in reducing recurrence of cancer; (9) use in reducing metastasis of tumor or cancer. For example, without being bound by theory, decreasing FOXO4 activity (e.g., via administration of an shRNA or a FOXO4 blocking peptide) can sensitize various cancer cells to chemotherapy, and can thus prevent relapses of cancer in vivo and improve survival. In some embodiments, any of the agents that inhibit FOXO4, the peptides or compositions provided herein, and at least one other chemotherapeutic agent are administered concurrently or sequentially. In some embodiments, any of the agents that inhibit FOXO4, the peptides or compositions provided herein is administered before or after radiation therapy or administration of at least one other chemotherapeutic agent.

In some embodiments, there is provided a method of inducing apoptosis of cells (or removing or killing cells such as senescent cells or cancer cells, or inducing apoptosis of senescent cells or cancer cells) (e.g., in an individual such as human) comprising contacting the cells with an effective amount of any one (or at least one) of the agents, peptides or compositions provided herein. Also provided are methods of treating an age-related disease or pathology or a symptom thereof (e.g., reducing or alleviating one or more symptoms of an age-related disease) comprising administering an effective amount of any one (or at least one) of the agents, peptides or compositions provided herein. Also provided are methods of treating a disease associated with senescent cells (e.g. accumulation of senescent cells) or pathology or a symptom thereof (e.g., reducing or alleviating one or more symptoms of a disease associated with senescent cells) (e.g., in an individual such as human) comprising administering an effective amount of any one (or at least one) of the agents, peptides or compositions provided herein. In some embodiments, the age-related disease or senescent cell-related disease and/or their pathology may be any one or more of Alzheimer's disease, Huntington's disease, diseases associated with cataracts, atherosclerosis, chronic obstructive pulmonary disease (COPD), emphysema, diabetic ulcer, kyphosis, herniated intervertebral discs, osteoarthritis, osteoporosis, Parkinson's disease, renal disease, renal failure, or sarcopenia.

In some embodiments, any of the agents that inhibit FOXO4, the peptides or compositions provided herein, and at least one other chemotherapeutic agent are administered concurrently or sequentially. In some embodiments, any of the agents that inhibit FOXO4, the peptides or compositions provided herein is administered before or after radiation therapy or administration of at least one other chemotherapeutic agent.

In some embodiments, any of the agents that inhibit FOXO4 (e.g., hairpin-based RNA) or the peptides provided herein may be delivered by liposomes or cell permeable peptide (“CPP”) (e.g., MPG, PEP-1, CADY, octa-arginine). In some embodiments, the CPP is fused to a peptide provided herein.

Agents that inhibit FOXO4 (e.g., peptides) can be made using methods such as methods disclosed in the Examples herein. Peptides described herein may be made by expression of nucleic acids encoding the peptides in host cells, or by chemical synthesis. Agents that inhibit FOXO4 (e.g., peptides) can be isolated or purified using methods known in the field.

Also provided herein are nucleic acids (e.g., isolated nucleic acids) encoding any one the peptides provided herein. Also provided herein are vectors (e.g., expression vectors) comprising any one of the nucleic acids provided herein. Also provided herein are cells (e.g., host cells) comprising any one of the vectors or nucleic acids provided herein.

Also provided herein are conjugates comprising an agent that inhibits FOXO4 (e.g., a peptide provided herein). Conjugates or other formulations may be used to extend half-life of FOXO4 inhibitory agents in vivo. In some embodiments, there is provided a conjugate comprising an agent that inhibits FOXO4 (e.g., a peptide provided herein) and a biocompatible polymer (e.g., a carrier protein, for example, an albumin such as human albumin) In some embodiments, there is provided a conjugate comprising an agent that inhibits FOXO4 (e.g., a peptide provided herein) and polyethylene glycol (PEG). In some embodiments, there is provided an agent that inhibits FOXO4 (e.g., a peptide provided herein) that is formulated in liposomes.

Methods of Using Agents that Inhibit FOXO4

Provided herein are methods of using agents that inhibit FOXO4, such as for treating cancer, for use as adjuvant therapy, inducing apoptosis of cells such as senescent cells or cancer cells, removing or killing senescent cells, treating an age-related disease or pathology or a symptom related thereof, conferring sensitivity of cells to radiation or chemotherapeutic agent(s), and/or uses in combination with radiation therapy and/or other chemotherapeutic agent(s). Any method provided herein may be conducted in an individual such as human.

In some embodiments, there is provided a method of treating cancer in an individual comprising administering to the individual an effective amount of an agent that inhibits FOXO4 (e.g., human FOXO4), wherein the agent is used as an adjuvant therapy. Also provided herein are methods of conferring sensitivity to chemotherapy in cancer cells (e.g., in an individual such as human) comprises contacting the cancer cells with an effective amount of an agent that inhibits FOXO4. Also provided herein are methods of treating cancer in an individual comprising administering to the individual an effective amount of (a) an agent that inhibits FOXO4; and (b) at least one other chemotherapeutic agent. Also provided herein are methods of treating cancer (e.g., non-melanoma cancer) in an individual comprising administering to the individual an effective amount of an agent that inhibits FOXO4.

In some embodiments, there is provided a method of treating cancer in an individual comprising administering to the individual an effective amount of (a) a peptide comprising an amino acid sequence that has at least 80% identity to a fragment in the DNA binding domain of FOXO4 (e.g., a fragment of SEQ ID NO:2); and/or (b) a peptide comprising an amino acid sequence that has at least 80% identity to a fragment in the C-terminal region of FOXO4 (e.g., a fragment of SEQ ID NO:3), wherein the peptide of (a) and peptide of (b) are used as an adjuvant therapy. Also provided herein are methods of conferring sensitivity to chemotherapy in cancer cells (e.g., in an individual such as human) comprises contacting the cancer cells with an effective amount of (a) a peptide comprising an amino acid sequence that has at least 80% identity to a fragment in the DNA binding domain of FOXO4 (e.g., a fragment of SEQ ID NO:2); and/or (b) a peptide comprising an amino acid sequence that has at least 80% identity to a fragment in the C-terminal region of FOXO4 (e.g., a fragment of SEQ ID NO:3). Also provided here are methods of treating cancer in an individual comprising administering to the individual an effective amount of (a) a peptide comprising an amino acid sequence that has at least 80% identity to a fragment in the DNA binding domain of FOXO4 (e.g., a fragment of SEQ ID NO:2); (b) a peptide comprising an amino acid sequence that has at least 80% identity to a fragment in the C-terminal region of FOXO4 (e.g., a fragment of SEQ ID NO:3); and/or (c) at least one other chemotherapeutic agent. Also provided here are methods of treating cancer in an individual comprising administering to the individual an effective amount of (a) a peptide comprising an amino acid sequence that has at least 80% identity to a fragment in the DNA binding domain of FOXO4 (e.g., a fragment of SEQ ID NO:2); and/or (b) a peptide comprising an amino acid sequence that has at least 80% identity to a fragment in the C-terminal region of FOXO4 (e.g., a fragment of SEQ ID NO:3), wherein the cancer is not melanoma.

In some embodiments, there is provided a method of treating cancer in an individual comprising administering to the individual an effective amount of any one (or at least one) of the agents, peptides or compositions provided herein. Also provided are methods of treating cancer in an individual comprising administering to the individual an effective amount of any one (or at least one) of the agents, peptides or compositions provided herein, wherein the peptide or composition is used as adjuvant therapy. Also provided are methods of conferring sensitivity to radiation in cancer cells (e.g., in an individual such as human) comprises contacting the cancer cells with an effective amount of any one (or at least one) of the agents, peptides or compositions provided herein. Also provided are methods of treating cancer in an individual comprising administering to the individual an effective amount of any one (or at least one) of the agents, peptides or compositions provided herein, wherein the peptide or composition is used in combination with radiation therapy. Also provided are methods of conferring sensitivity to chemotherapy (or chemotherapeutic agent) in cancer cells (e.g., in an individual such as human) comprises contacting the cancer cells with an effective amount of any one (or at least one) of the agents, peptides or compositions provided herein. Also provided are methods of treating cancer in an individual comprising administering to the individual an effective amount of any one (or at least one) of the agents, peptides or compositions provided herein, wherein the peptide or composition is used in combination with at least one other chemotherapeutic agent. Also provided herein are methods of inducing apoptosis of cells (or removing or killing cells such as senescent cells or cancer cells, or inducing apoptosis of senescent cells or cancer cells) (e.g., in an individual such as human) comprising contacting the cells with an effective amount of any one (or at least one) of the agents, peptides or compositions provided herein.

In some embodiments, there is provided a method of treating cancer in an individual comprising administering to the individual an effective amount of (a) a peptide comprising an amino acid sequence that has at least 80% identity to a fragment in the DNA binding domain of FOXO4 (e.g., a fragment in SEQ ID NO:2); and/or (b) a peptide comprising an amino acid sequence that has at least 80% identity to a fragment in the C-terminal region of FOXO4 (e.g., a fragment in SEQ ID NO:3). Also provided herein are methods of treating cancer in an individual comprising administering to the individual an effective amount of (a) at least one peptide comprising an amino acid sequence that has at least 80% identity to a fragment in the DNA binding domain of FOXO4 (e.g., a fragment in SEQ ID NO:2 such as SRRNAWGNQSYAELIS or PRKGGSRRNAWGNQSYAELISQAIESAPEKRLTL); and/or (b) at least one peptide comprising an amino acid sequence that has at least 80% identity to a fragment in the C-terminal region of FOXO4 (e.g., a fragment in SEQ ID NO:3 such as LECDMDNIISDLMDEGEGLDF or PQDLDLDMYMENLECDMDNIISDLMDEGEGLDF), wherein the peptide(s) are used as adjuvant therapy.

In some embodiments, there is provided a method of conferring sensitivity to radiation in cancer cells (e.g., in an individual such as human) comprises contacting the cancer cells with an effective amount of (a) at least one peptide comprising an amino acid sequence that has at least 80% identity to a fragment in the DNA binding domain of FOXO4 (e.g., a fragment in SEQ ID NO:2 such as SRRNAWGNQSYAELIS or PRKGGSRRNAWGNQSYAELISQAIESAPEKRLTL); and/or (b) at least one peptide comprising an amino acid sequence that has at least 80% identity to a fragment in the C-terminal region of FOXO4 (e.g., a fragment in SEQ ID NO:3 such as LECDMDNIISDLMDEGEGLDF or PQDLDLDMYMENLECDMDNIISDLMDEGEGLDF). Also provided herein are methods of treating cancer in an individual comprising administering to the individual an effective amount of (a) at least one peptide comprising an amino acid sequence that has at least 80% identity to a fragment in the DNA binding domain of FOXO4 (e.g., a fragment in SEQ ID NO:2 such as SRRNAWGNQSYAELIS or PRKGGSRRNAWGNQSYAELISQAIESAPEKRLTL); and/or (b) at least one peptide comprising an amino acid sequence that has at least 80% identity to a fragment in the C-terminal region of FOXO4 (e.g., a fragment in SEQ ID NO:3 such as LECDMDNIISDLMDEGEGLDF or PQDLDLDMYMENLECDMDNIISDLMDEGEGLDF), wherein the peptide(s) are used in combination with radiation therapy. Also provided herein are methods of conferring sensitivity to chemotherapy (or chemotherapeutic agent) in cancer cells (e.g., in an individual such as human) comprises contacting the cancer cells with an effective amount of (a) at least one peptide comprising an amino acid sequence that has at least 80% identity to a fragment in the DNA binding domain of FOXO4 (e.g., a fragment in SEQ ID NO:2 such as SRRNAWGNQSYAELIS or PRKGGSRRNAWGNQSYAELISQAIESAPEKRLTL); and/or (b) at least one peptide comprising an amino acid sequence that has at least 80% identity to a fragment in the C-terminal region of FOXO4 (e.g., a fragment in SEQ ID NO:3 such as LECDMDNIISDLMDEGEGLDF or PQDLDLDMYMENLECDMDNIISDLMDEGEGLDF). Also provided herein are methods of treating cancer in an individual comprising administering to the individual an effective amount of (a) at least one peptide comprising an amino acid sequence that has at least 80% identity to a fragment in the DNA binding domain of FOXO4 (e.g., a fragment in SEQ ID NO:2 such as SRRNAWGNQSYAELIS or PRKGGSRRNAWGNQSYAELISQAIESAPEKRLTL); and/or (b) at least one peptide comprising an amino acid sequence that has at least 80% identity to a fragment in the C-terminal region of FOXO4 (e.g., a fragment in SEQ ID NO:3 such as LECDMDNIISDLMDEGEGLDF or PQDLDLDMYMENLECDMDNIISDLMDEGEGLDF), wherein the peptide(s) are used in combination with one other chemotherapeutic agent.

In some embodiments, there is provided a method of inducing apoptosis of cells (or removing or killing cells such as senescent cells or cancer cells, or inducing apoptosis of senescent cells or cancer cells) (e.g., in an individual such as human) comprising contacting the cells with an effective amount of any one (or at least one) of the agents, peptides or compositions provided herein. Also provided are methods of treating an age-related disease or pathology or a symptom thereof (e.g., reducing or alleviating one or more symptoms of an age-related disease) comprising administering an effective amount of any one (or at least one) of the agents, peptides or compositions provided herein. In some embodiments, the age-related disease or senescent cell-related disease and/or their pathology may be any one or more of Alzheimer's disease, Huntington's disease, diseases associated with cataracts, atherosclerosis, chronic obstructive pulmonary disease (COPD), emphysema, diabetic ulcer, kyphosis, herniated intervertebral discs, osteoarthritis, osteoporosis, Parkinson's disease, renal disease, renal failure, or sarcopenia. For example, there is provided a method of inducing apoptosis of senescent cells (or killing senescent cells, or inducing apoptosis of cells (e.g., senescent cells or cancer cells)) (e.g., in an individual such as human) comprising contacting the cells with an effective amount of (a) at least one peptide comprising an amino acid sequence that has at least 80% identity to a fragment in the DNA binding domain of FOXO4 (e.g., a fragment in SEQ ID NO:2 such as SRRNAWGNQSYAELIS or PRKGGSRRNAWGNQSYAELISQAIESAPEKRLTL); and/or (b) at least one peptide comprising an amino acid sequence that has at least 80% identity to a fragment in the C-terminal region of FOXO4 (e.g., a fragment in SEQ ID NO:3 such as LECDMDNIISDLMDEGEGLDF or PQDLDLDMYMENLECDMDNIISDLMDEGEGLDF). In some embodiments, there is provided a method of treating an age-related disease or pathology or a symptom thereof (e.g., reducing or alleviating one or more symptoms of an age-related disease) comprising administering an effective amount of (a) at least one peptide comprising an amino acid sequence that has at least 80% identity to a fragment in the DNA binding domain of FOXO4 (e.g., a fragment in SEQ ID NO:2 such as SRRNAWGNQSYAELIS or PRKGGSRRNAWGNQSYAELISQAIESAPEKRLTL); and/or (b) at least one peptide comprising an amino acid sequence that has at least 80% identity to a fragment in the C-terminal region of FOXO4 (e.g., a fragment in SEQ ID NO:3 such as LECDMDNIISDLMDEGEGLDF or PQDLDLDMYMENLECDMDNIISDLMDEGEGLDF).

In some embodiments of any of the methods provided herein, the method further comprises radiation therapy (such as ionizing radiation or X-ray). In some embodiments of any of the methods provided herein, the method further comprises administration of at least one other chemotherapeutic agent.

An exemplary and non-limiting list of chemotherapeutic agents contemplated is provided herein. Suitable chemotherapeutic agents include, for example, vinca alkaloids, agents that disrupt microtubule formation (such as colchicines and its derivatives), anti-angiogenic agents, therapeutic antibodies, transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof) and other standard chemotherapeutic agents well recognized in the art.

In certain embodiments, at least one other chemotherapeutic agent is a therapeutic antibody, a topoisomerase inhibitor, an antimetabolite, a platinum-based agent, an alkylating agent, a tyrosine kinase inhibitor, an Anthracycline antibiotic, an anti-angiogenic agent, or a vinca alkaloid. In some embodiments, the at least one other chemotherapeutic agent used herein is a RAF inhibitor (e.g., RAF265), BRAFV600E inhibitor (e.g., PLX4032), or MEK inhibitor (e.g., U0126, AZD6244 or selumetinib). In some embodiments, the at least one other chemotherapeutic agent is 5-FU, cisplatin, dacarbazine, RAF265, PLX4032, AZD6244 (selumetinib), gemcitabine, capecitabine, methotrexate (anti-folic acid), vinblastine, doxorubicin, or mitoxantrone.

Examples of cancer include but are not limited to, carcinoma, including adenocarcinoma, lymphoma, blastoma, melanoma, and sarcoma. More particular examples of such cancers include squamous cell cancer, small-cell lung cancer, non-small cell lung cancer, lung adenocarcinoma, lung squamous cell carcinoma, gastrointestinal cancer, Hodgkin's and non-Hodgkin's lymphoma, pancreatic cancer, glioblastoma, cervical cancer, glioma, ovarian cancer, liver cancer such as hepatic carcinoma and hepatoma, bladder cancer, breast cancer, colon cancer, colorectal cancer, endometrial or uterine carcinoma, salivary gland carcinoma, kidney cancer such as renal cell carcinoma and Wilms' tumors, basal cell carcinoma, melanoma, prostate cancer, thyroid cancer, testicular cancer, esophageal cancer, and various types of head and neck cancer.

In some embodiments, the cancer is melanoma. In some embodiments, the cancer is not melanoma. In some embodiments, the cancer is skin cancer (such as melanoma), mammary cancer, breast cancer, prostate cancer, pancreatic cancer, ovarian cancer, glioblastoma, renal cancer, or bladder cancer. In some embodiments, the cancer does not comprise mutation in p53. (e.g., the cancer is wildtype for p53).

An agent that inhibits FOXO4 or a composition provided herein may be formulated for administration by intraperitoneal, intravenous, subcutaneous, and intramuscular injections, and other forms of administration such as oral, mucosal, via inhalation, sublingually, etc. In some embodiments, an agent that inhibits FOXO4 or a composition provided herein is administered by any means known in the art, such as intraperitoneally, intravenously, intramuscularly, subcutaneously, intrathecally, intraventricularly, orally, enterally, parenterally, intranasally, dermally, sublingually, by inhalation, local or systemic administration, including injection, oral administration, particle gun or catheterized administration, or topical administration. An agent that inhibits FOXO4 or a composition provided herein may be formulated to extend half lives in vivo, such as by forming conjugates with biocompatible polymer (e.g., a carrier protein, for example, an albumin such as human albumin) or polyethylene glycol (PEG). An agent that inhibits FOXO4 or a composition provided herein may also be delivered by using liposomes.

An agent that inhibits FOXO4 or a composition provided herein may be used in combination with another treatment such as radiation, chemotherapy, or surgery, concurrently or sequentially. For example, an agent that inhibits FOXO4 or a composition provided herein may be used concurrently with or subsequent to another chemotherapeutic agent. An agent that inhibits FOXO4 or a composition provided herein may be used before or after another chemotherapeutic agent.

### Articles of Manufacture and Kits

The invention also provides kits and articles of manufactures for use in the instant methods. Kits of the invention include one or more containers comprising an agent that inhibits FOXO4 as described herein and instructions for use in accordance with any of the methods of the invention described herein.

In some embodiments, these instructions comprise a description of administration of the agent that inhibits FOXO4 for treating cancer, for use as adjuvant therapy, inducing apoptosis of cells such as senescent cells or cancer cells, killing or removing cells such as senescent cells or cancer cells, conferring sensitivity of cells to radiation or chemotherapeutic agent(s), and/or uses in combination with radiation therapy and/or other chemotherapeutic agent(s). The kit may further comprise a description of selecting an individual suitable for treatment based on identifying whether that individual has the disease and the stage of the disease.

The instructions relating to the use of the agents that inhibit FOXO4 generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating a cancer or other uses described herein. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an agent or peptide described herein. The container may further comprise a second pharmaceutically active agent.

Kits may optionally provide additional components such as buffers and interpretive information. Normally, the kit comprises a container and a label or package insert(s) on or associated with the container.

The following are examples of the methods and compositions of the invention. It is understood that various other embodiments may be practiced, given the general description provided above.

### EXAMPLES

### Example 1

### Studying the Roles of FOXO4 in Apoptosis

This example describes one method to interfere with the survival of (A) senescent and (B) cancer cells by forcing a shift in their response to stress toward apoptosis.

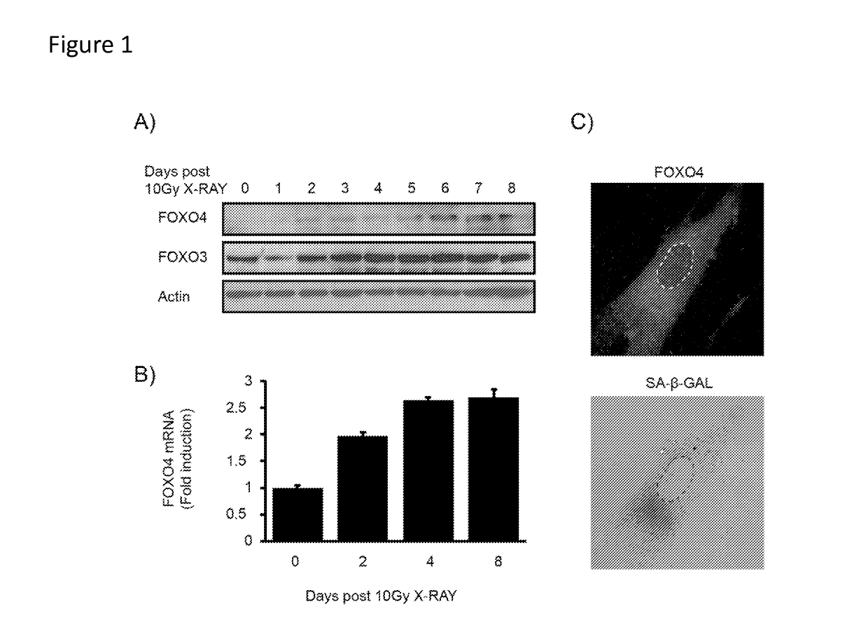

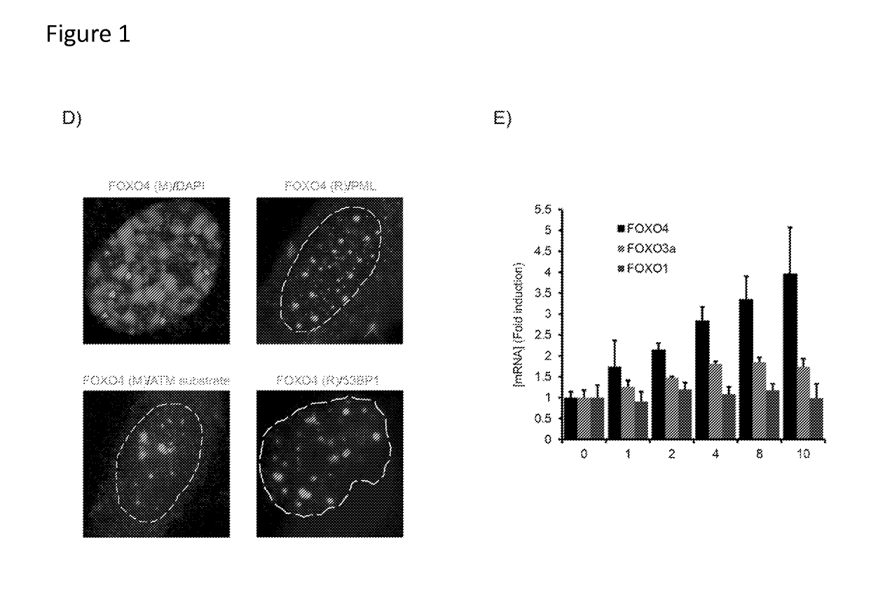

FOXO4 is a molecular pivot that decides whether damaged cells undergo senescence or apoptosis. Some references state that it is unclear why some cells undergo apoptosis whereas others enter senescence in response to identical stimuli. FOXO transcription factors are negatively regulated by growth factor signaling, but, as we and others have shown, they can also be activated by oxidative stress (Brunet, A. et al., Science 303, 2011-2015 (2004); de Keizer, P. L. et al., Cancer Res 70, 8526-8536 (2010); Essers, M. A. et al., EMBO J. 23, 4802-4812 (2004)). Constitutive foxo1^{-/-} mice are embryonic lethal and foxo3^{-/-} mice show reproductive deficiencies, foxo4^{-/-} mice do not harbor a significantly defective phenotype (Hosaka, T. et al., Proc.Natl.Acad.Sci.U.S.A 101, 2975-2980 (2004); Castrillon, D. H. et al., Science 301, 215-218 (2003)). Individual conditional somatic foxo3^{-/-} mice show a slightly shortened lifespan, whereas conditional somatic foxo1^{-/-} and foxo4^{-/-} do not (Paik, J. H. et al., Cell 128, 309-323 (2007)). Somatic triple foxo-1,3,4−/− mice show an increase in lymphoma thus indicating that in this respect FOXOs are functionally redundant (Paik, J. H. et al., Cell 128, 309-323 (2007)). Notably however, single somatic foxo4^{-/-} mice do not show any shortened lifespan, nor any changes in tumor-free survival. We observed that while FOXO1 and FOXO3 levels remain largely unchanged, FOXO4 mRNA and protein expression rise significantly in response to senescence-inducing levels of DNA damage (FIG. 1a+b).

Senescence caused by ionizing radiation (XRAY)-induced DNA damage is characterized by the formation of persistent nuclear foci termed DNA-SCARS (DNA Segments with Chromatin Alterations Reinforcing Senescence), which are required for the growth arrest (Rodier, F. et al., J Cell Sci 124, 68-81 (2011)). DNA-SCARS are adjacent to PML bodies, which contain a host of proteins involved in senescence. We observed that under senescence-inducing conditions FOXO4 localizes to punctate structures (FIG. 1c). These FOXO4 foci turned out to be PML bodies and indeed FOXO4 punctae lie adjacent to 53BP1 and phosphorylated ATM substrates (two markers of DNA-SCARS) and are mutually exclusive with heterochromatin (FIG. 1d).

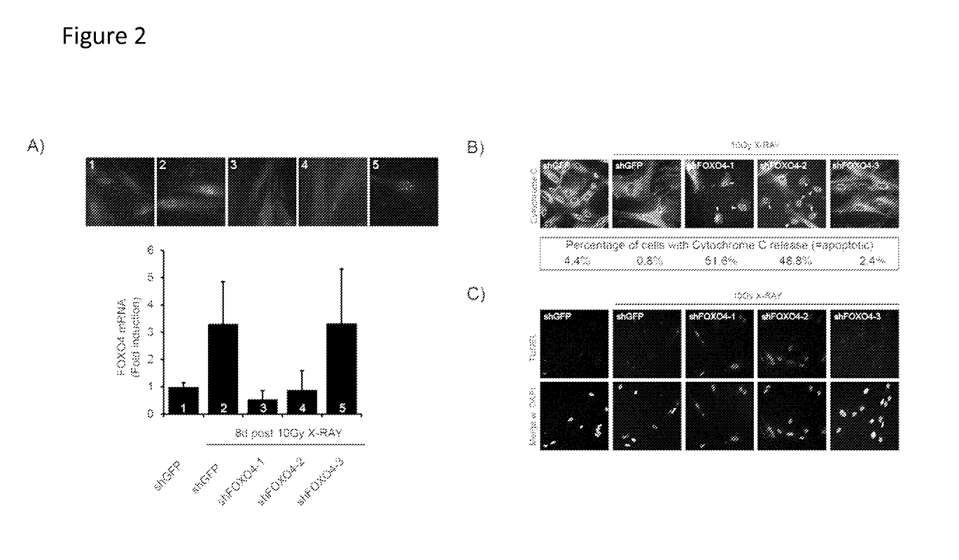

Under these DNA-damaging conditions loss of FOXO4 expression using stable short hairpin-based RNA interference (shRNA) induced apoptosis instead of senescence (FIG. 2a-c). This result established that FOXO4 is a pivotal factor in the molecular decision of whether cells senesce or apoptose in response to genotoxic stress. Moreover, this result identifies a vulnerability that can be exploited for therapeutic removal of senescent cells.

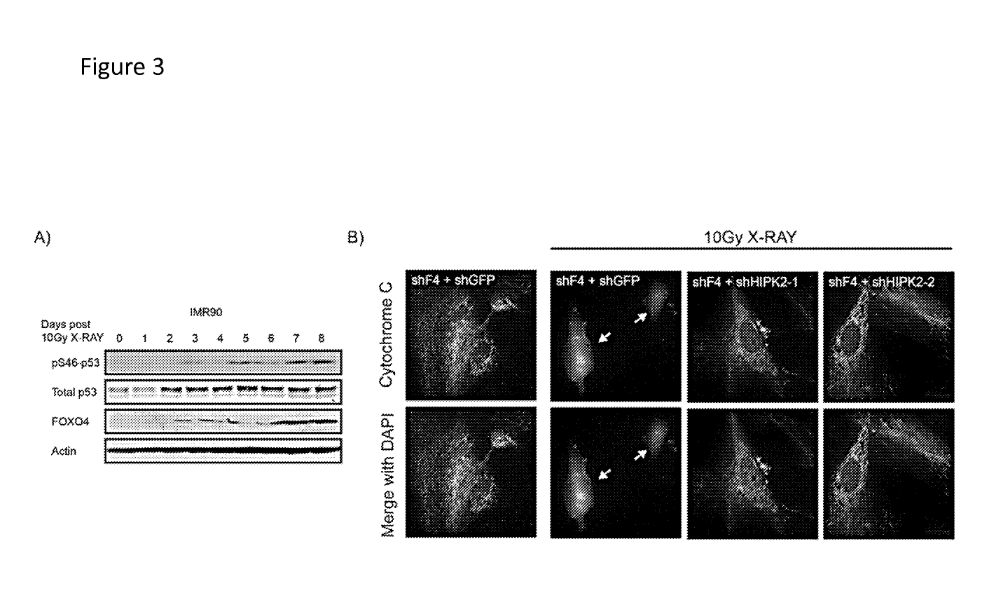

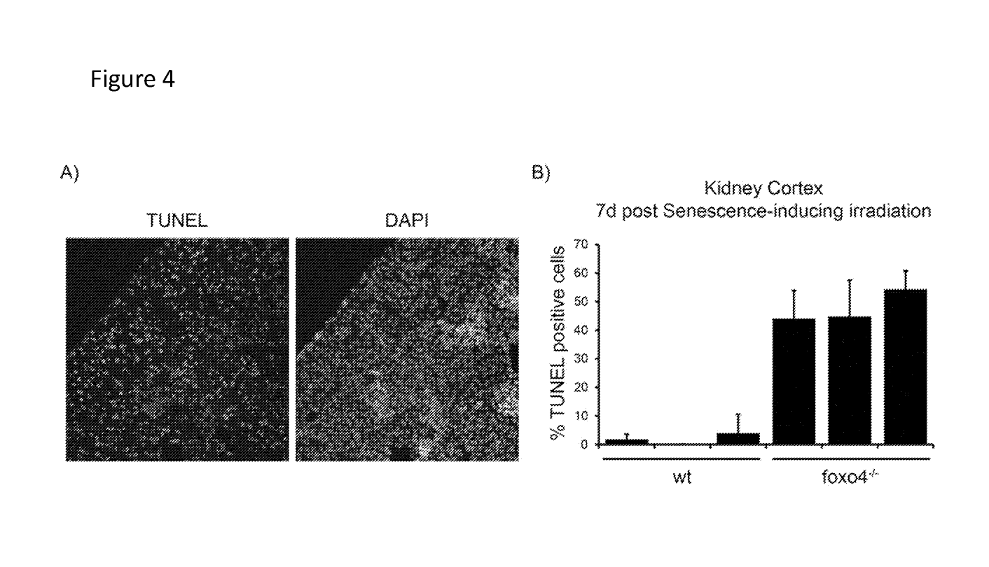

The mechanism by which FOXO4 restrains apoptosis in favor of senescence involves its physical association with the p53 tumor suppressor protein. p53 is well known to regulate cell fate after DNA damage (Rodier, F. et al., Nucleic Acids Res 35, 7475-7484 (2007)) and is a major component of DNA-SCARS (Rodier, F. et al., Nat. Cell Biol. 11, 973-979 (2009)). p53 can induce senescence as well as an apoptosis, depending on its post-translational modifications and its interaction partners (Vousden, K. H. et al., Nat. Rev. Mol. Cell Biol. 8, 275-283 (2007)). When phosphorylated on Ser46, p53 strongly favors apoptosis over cell cycle arrest (Bulavin, D. V. et al., EMBO J. 18, 6845-6854 (1999)). However, Ser46 is phosphorylated in response to several senescence-inducing stimuli, including activated oncogenes (Feng, L. et al., Cell Cycle 5, 2812-2819 (2006); Bischof, O. et al., EMBO J. 21, 3358-3369 (2002)). One query is how apoptosis is restrained in these cases. We observed that under DNA damaging conditions Ser46-phosphorylation of p53 becomes elevated (FIG. 3a) and that interference with the HIPK2 kinase, which is responsible for Ser46-phosphorylation (Dauth, I. et al., Cancer Res 67, 2274-2279 (2007)), impairs the apoptotic response caused by FOXO4 depletion (FIG. 3b). Thus FOXO4 restrains apoptosis in senescent cells by repressing the apoptotic arm of p53 signaling in favor of senescence (FIG. 4).

### A) Removal of Senescent Cells by Anti-FOXO4 Therapy for Treatment of Age-Related Pathologies

The use of FOXO4 inhibition to switch senescent cells toward apoptosis has the following potential therapeutic benefits:

1) Reducing the detrimental effects of senescent cells on tissue homeostasis. The paracrine activities of the SASP have detrimental effects on the function of neighboring cells, and can create a local, and possibly systemic, milieu that can drive many age-related pathologies, including cancer (Coppe, J. P. et al., Annu Rev Pathol 5, 99-118 (2010); Freund, A. et al., Trends Mol Med 16, 238-246 (2010); Campisi, J. et al., Semin Cancer Biol 21, 354-359 (2011)). As senescent cells accumulate with age, their removal can prevent the disruption of tissue homeostasis and development of age-related disease.

2) Reducing inflammation after chemo/radio-therapy (cancer supportive care). A major side effect of treating cancer patients with chemo/radiotherapy is the ensuing inflammation. Chemo/radiotherapies also induce cellular senescence and the SASP (Coppe, J. P. et al., PLoS. Biol. 6, 2853-2868 (2008); Rodier, F. et al., Nat. Cell Biol. 11, 973-979 (2009)). Clearing senescent cells after chemo/radio therapy by FOXO4 inhibition would considerably reduce these detrimental off-target effects.

3) Senescent cells are present at sites of age-related pathologies. Further, the elimination of senescent cells from a mouse model of premature aging prevented the development of cataracts and sarcopenia (Baker, D. J. et al., Nature 479, 232-236 (2011)). Thus, causing senescent cells to undergo apoptosis by inhibiting FOXO4 has therapeutic potential for treating a wide range of age-related pathologies and/or pathologies associated with the senescent cells.

### B) Anti-FOXO Therapy as Adjuvant Treatment for the Removal of Cancer Cells

The acquisition of new mutations in senescent cells can result in escape from the growth arrest and progression toward malignancy. Given that FOXO4 interference induces a shift in the senescence response towards apoptosis, anti-FOXO4 therapy complement and/or optimize current chemo/radiotherapies.

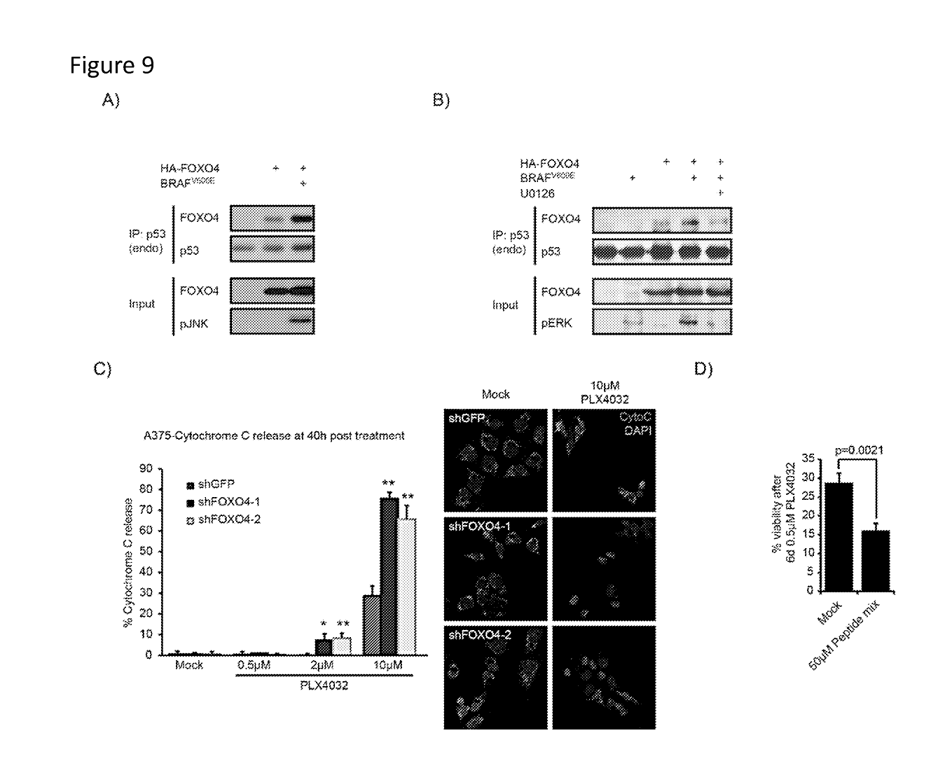

We tested whether interference with FOXO4 expression or activity sensitized metastatic melanoma cells to chemotherapy. Metastatic melanoma is a lethal skin cancer for which there are few effective treatments. Oncogenic BRAF mutations (typically V600E) are found in ˜7% of all human tumors, with an especially high occurrence in melanoma (˜70%) (Davies, H. et al., Nature 417, 949-954 (2002)). We previously showed that BRAFV600E promotes FOXO4 activation, thereby promoting senescence in a p21^{Cip1}-mediated fashion (de Keizer, P. L. et al., Cancer Res 70, 8526-8536 (2010)). Additionally, we now observed that FOXO4 physically interacts with p53, a well-known activator of p21^{Cip1} transcription, in a manner that was enhanced by active BRAF^{V600E}-signaling (FIG. 9a). Interference with FOXO4 expression or the interaction with p53 may sensitize BRAF^{V600E}-driven melanoma cells towards apoptosis by interfering with this senescence pathway.

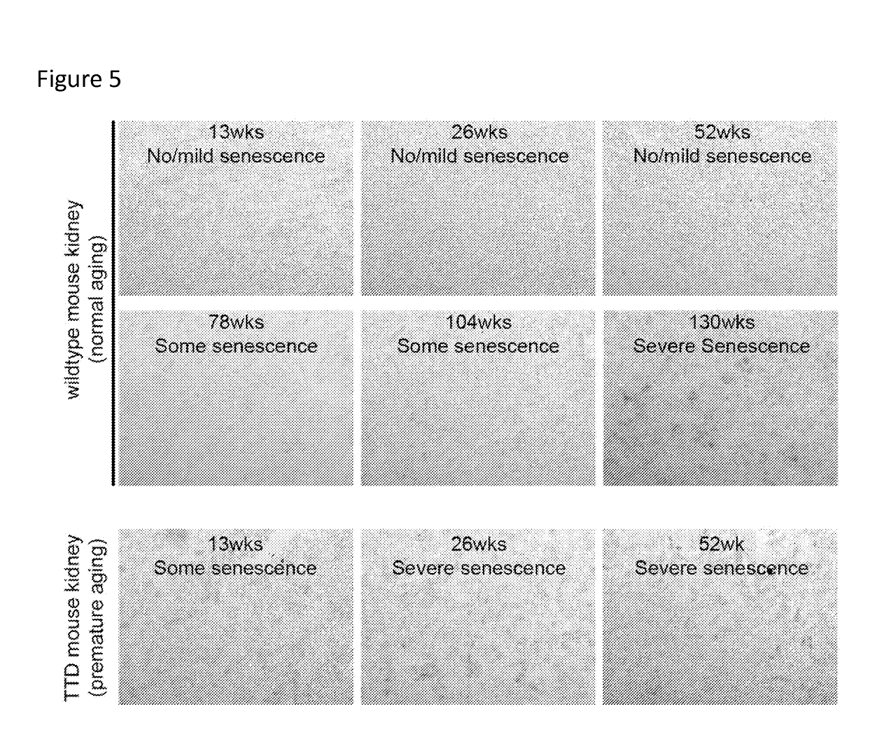

PLX4032 (Vemurafenib)) is a potent specific inhibitor of the V600E-mutated BRAF oncogene. Major improvements in tumor-free survival rates have been reported for patients with metastatic melanoma who were treated with PLX4032 (Bollag, G. et al., Nature 467, 596-599 (2010); Sosman, J. A. et al., N Engl J Med 366, 707-714 (2012)). PLX4032 was shown to trigger apoptosis in BRAF^{V600E}-driven melanoma cells such as A375 (Sala, E. et al., Mol Cancer Res 6, 751-759 (2008)). Unfortunately, not all BRAF^{V600E}-mutated tumors are equally sensitive to PLX4032, and PLX4032-resistant tumors tend to recur (Johannessen, C. M. et al., Nature 468, 968-972 (2010)). Thus, there is a need to enhance the sensitivity of melanomas to PLX4032. We observed that knockdown of FOXO4 expression in BRAF^{V600E} driven malignant A375 melanoma cells strongly sensitized them to PLX4032 (FIG. 5b).

Interference with the FOXO4-p53 interaction may lead to a similar result. The in vitro interaction domain of FOXO3a and p53 has been mapped by NMR and was shown to occur in two independent regions in FOXO3a, one in the DNA binding domain and one in the C-terminal transactivation domain (Wang, F. et al., J Mol Biol 384, 590-603 (2008)). Based on sequence similarity, we designed two FOXO4-p53 blocking peptides based on the interaction motifs. A mixture of both peptides sensitized A375 cells to PLX4032 in a dose-dependent manner (FIG. 9c).

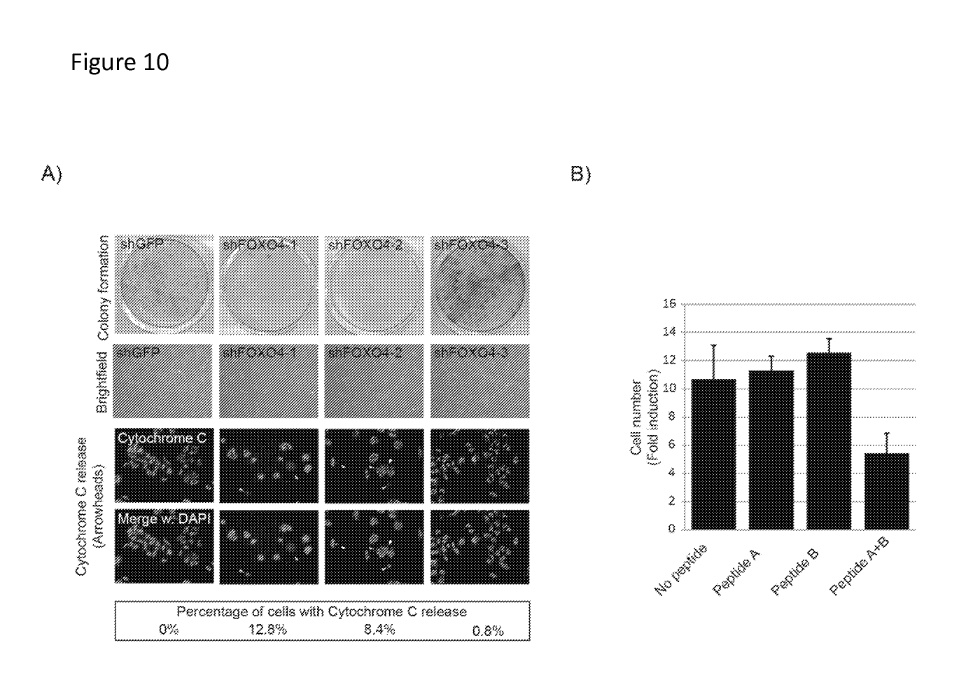

Resistance to PLX4032 is in part due to the hyperactivating mutation of NRAS, a different oncogene that is frequently mutated in melanoma (Nazarian, R. et al., Nature 468, 973-977 (2010)). Further underscoring the potential clinical usefulness of our method, at least one NRAS-mutated melanoma cell-line, D04, could spontaneously be induced to undergo apoptosis by FOXO4 interference (either through RNAi or FOXO4-p53 blocking peptides; FIG. 10a+b). Thus FOXO4 inhibition could significantly improve survival of melanoma patient not only by sensitizing BRAF^{V600E}-mutated primary tumor/metastases to PLX4032, but also by limiting the recurrence of PLX4032-resistant tumors.

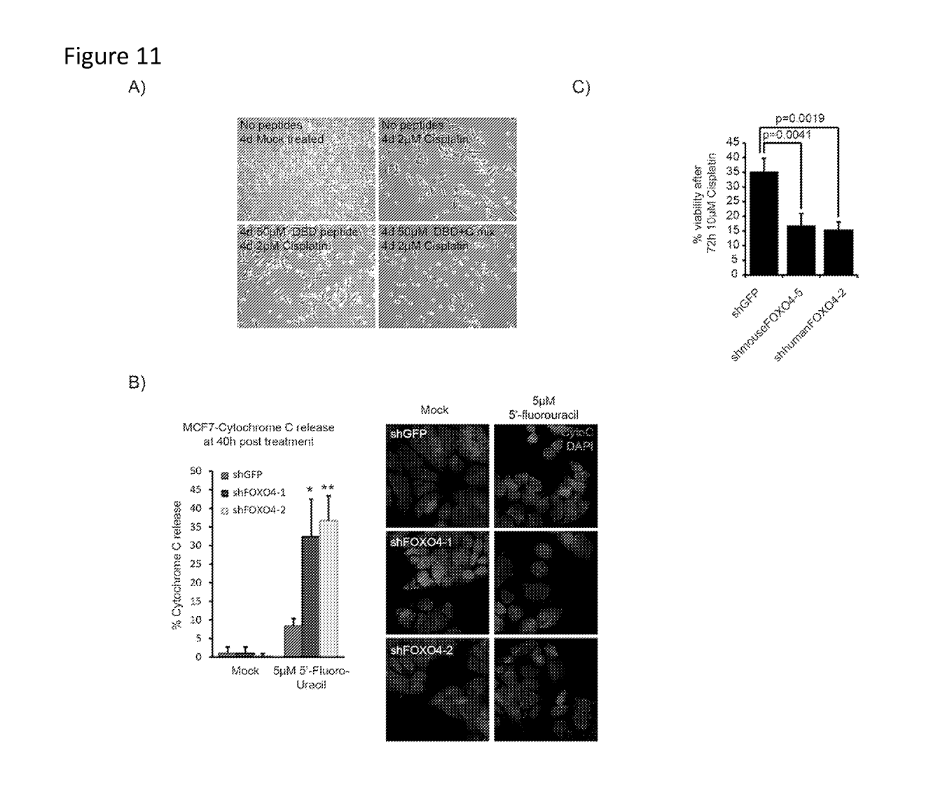

The use of FOXO4 inhibition need not be restricted to one chemotherapeutic agent, nor one type of cancer. Preliminary data showed that in addition to PLX4032, A375 melanoma cells incubated with the p53-FOXO4 blocking peptide mix were extra sensitive to Cisplatin which is used not only useful to treat melanoma (Li, W. et al., Oncogene, (2011)), but a variety of cancers (Lorch, J. H. et al., Lancet Oncol 12, 153-159 (2011); Mitsudomi, T. et al., Lancet Oncol 11, 121-128 (2010); Goa, A. K. et al., Indian J Med Paediatr Oncol 31, 76-78 (2010)) (FIG. 11a). Furthermore, we observed that FOXO4 knockdown sensitized MCF7 mammary carcinoma cells to another drug frequently used to treat these tumors-5-fluorouracyl (Tan, S. H. et al., Clin Cancer Res 14, 8027-8041 (2008)) (FIG. 11b). The targeted anti-FOXO4 therapy in vivo is also studied.

In summary, FOXO4 can be a target that is implicated in the survival of senescent cells and certain cancer cells. Targeting FOXO4 expression may be clinically efficacious as a treatment or adjuvant for melanoma and other cancers, delay the recurrence of chemotherapy-resistant tumors, and reduce metastases (if given before/at the time of chemotherapy).

### Experimental Methods

FIG. 1A: 3×10⁶ Normal Human IMR90 fibroblasts were plated in 6 cm dishes and the next day exposed to 10Gy ionizing radiation (XRAY) or left untreated. At the indicated time-points protein samples were prepared by collecting the cells in 1× Laemli Sample buffer. The indicated proteins were detected by western blot using antibodies against FOXO4 (#9472, Cell Signaling), FOXO3a (#07-702, Upstate/Millipore) and Tubulin (B-5-1-2, Sigma-Aldrich). FIG. 1B: 10⁴ Normal human HCA2 fibroblasts were plated in 96-well plates in triplicate per condition and the next day exposed to 10Gy ionizing radiation (XRAY) or left untreated. At the indicated time-points mRNA and subsequent cDNA samples were collected using the Cells-to-Ct kit from Ambion according to the manufacturer's instructions. Real-time QPCR was subsequently performed using the Universal Probe Library system from Roche with the following primers/probe combinations: For FOXO4: Probe 18, FOXO4-fwd: 5′-acgagtggatggtccgtact-3′, FOXO4-Rev: 5′-gtggcggatcgagttcttc-3′. For Tubulin: Probe 58, Tubulin-Fwd: 5′-cttcgtctccgccatcag-3′ and Tubulin-Rev 5′-ttgccaatctggacacca-3′. Changes in mRNA expression were calculated using the ΔΔCT method. FIG. 1C: 2×10⁵ IMR90 fibroblasts were plated on glass coverslips and the next day exposed to 10Gy ionizing radiation (XRAY) or left untreated. At 8 days post XRAY cells were washed twice in TBS on ice, fixed for 15 min in 4% formalin at 4° C., washed twice in TBS at room temperature (RT), washed once briefly in MilliQ at RT to remove residual TBS and incubated overnight at 37° C. in SA-β-Gal media containing 25.26 mM NA₂PO₄/7.37 mM Citric Acid buffer at pH6.0, 150 mM NaCl, 2 mM MgCl₂, 5 mM Potassium Ferricyanide, 5 mM Potassium Ferrocyanide, 0.1% X-GAL (in DMF). The next day the cells were washed twice with TBS on ice and refixed for 15 min with 4% formalin at 4° C. Subsequently the cells were processed for immunofluorescence and washed twice with TBS, permeabilized for 2 min with TBS-2% TX100, washed twice with TBS, quenched 10 min with TBS-50 mM Glycine, washed twice with TBS, blocked 30 min with TBS-0.2% w/v Gelatin. To detect FOXO4 the cells were incubated overnight with an antibody against FOXO4 (#9472, Cell Signaling) in TBS-Gel at 4° C., washed twice with TBS-Gel, incubated for two hours with an Alexa555-conjugated secondary antibody at room temperature and washed twice with TBS-Gel and once with TBS. The coverslips were mounted on microscope slides using ProlonGold with DAPI (Invitrogen). FIG. 1D: Similar experiment as in C), except no SA-β-Gal staining was performed. After treatment, the cells were fixed for 30 min in 4% formalin and processed as in C), using antibodies against FOXO4 (#9472, Cell Signaling), PML (sc-9862, Santa Cruz Biotechnology) and 53BP1 (05-726, Millipore). The ATM substrate antibody is from Cell signaling, #2851. FIG. 1E: Identical experiment as FIG. 1B, except in addition these probe/primer sets were used for FOXO1 and FOXO3a. For FOXO1: Probe, FOXO1-fwd: 5′-aagggtgacagcaacagctc-3′, FOXO1-Rev: 5′-ttctgcacacgaatgaacttg-3′. For FOXO3a: Probe 4481, FOXO3a-Fwd: 5′-gggttgtttcaatctaacagtcaa-3′ and FOXO3a-Rev 5′-caacattacggattgtgtagcc-3′.

FIG. 2A: HCA2 fibroblasts were infected with lentiviral particles containing a control shRNA-encoding vector targeted against a mature sequence in GFP (based on vector PLK0.1, Open Biosystems): 5′-CCAGCTTCAGTCAGCAGTTAT-3′ or two independent sequences in (human) FOXO4, shFOXO4-1: 5′-CCAGCTTCAGTCAGCAGTTAT-3′ and shFOXO4-2: 5′-CGTCCACGAAGCAGTTCAAAT-3′. Following selection for two days in 0.5 μg/ml Puromycin cells were plated and processed as in FIG. 1 to determine FOXO4 mRNA expression and FOXO4 localization by immunofluorescence. FIG. 2B: IMR90 fibroblasts were infected and treated as in A). Following selection 2×10⁵ cells were plated on coverslips and irradiated the next day. After irradiation the media was refreshed with media containing 20 μM of the general caspase inhibitor Q-VD-OPH (MP Bioscience). After three days the media was refreshed again with Q-VD-OPH and left for another two days. The cells were then processed for Cytochrome C localization as in FIG. 1c using an antibody against Cytochrome C (BD Pharmigen). The percentage of cells with released Cytochrome C staining (translocated from punctual mitochondrial staining to diffuse or absent staining) was determined for 100 cells per condition. FIG. 2C: Similar experiment as in B), except that apoptosis was addressed by TUNEL In Situ Cell Death Detection Kit at day 3 post irradiation according to the manufacturer's instructions (Roche).

FIG. 3A: IMR90 fibroblasts were treated and processed as in FIG. 1A) and protein expression was determined using antibodies against Ser46-phosphorylated p53 (2521S, Cell signaling), total p53 (D01, Santa Cruz Biotechnology), FOXO4 (#9472, Cell Signaling) and Alpha-Actin. FIG. 3B: IMR90 cells were infected as in FIG. 2A with lentiviral particles containing shFOXβ4-1, in combination with lentiviral particles containing shGFP or two shRNA targeted against HIPK2: shHIPK2-1 5′-CGAGTCAGTATCCAGCCCAAT-3′ and shHIPK2-2 5′-CGGGACAAAGACAACTAGGTT-3′. Cytochrome C-release was stained as in FIG. 2b.

Next, we addressed the function of FOXO4 in the regulation of senescence and apoptosis in vivo. To this extent we exposed wildtype mice and foxo4^{-/-} mice to a non-lethal dose of Ionizing Radiation that is known to induce senescence in mouse organs including kidneys, lung and skin (5Gy IR) (Le O N, Rodier F et al. Aging Cell. 2010 June; 9(3):398-409). Strikingly, exposure of foxo4^{-/-} mice, but not wildtype mice, to these levels of senescence-inducing IR resulted in strong induction of apoptosis in for instance the kidney cortex (FIG. 4). This underscores that also in vivo FOXO4 restrains apoptosis in favor of senescence.

Senescent cells are known to accumulate with age in humans and mice (Ressler S, Bartkova J Aging Cell. 2006 October; 5(5):379-89) (Yamakoshi K, Takahashi A et al. J. Cell Biol. 2009 Aug. 10; 186(3):393-407) (Burd C E, Sorrentino J A et al. Cell. 2013 Jan. 17;152(1-2):340-51). In addition to IR-induced senescence we therefore also addressed the effects of FOXO4 inhibition in naturally occurring senescence. Trichothiodystrophy (TTD) is a human genetic disorder characterized by premature aging (Tay CH Arch Dermatol 104 (1): 4-13) (Freedberg, et al. (2003). Fitzpatrick's Dermatology in General Medicine (6th ed.). McGraw-Hill. p. 501). A mouse model that mimics these progeroid features has been generated by mutation of the homologous DNA-damage Repair protein XPD, referred to as TTD mice (de Boer J, de Wit J et al. Mol. Cell. 1998 June; 1(7):981-90) (Vermeulen W, Bergmann E et al. Nat. Genet. 2000 November; 26(3):307-13). Where senescence became gradually apparent at around 78 weeks and older in kidneys of wildtype mice, kidneys of TTD mice already showed signs of senescence as soon as 13 weeks. Thus in this model for premature aging, senescence occurs early in life (FIG. 5).

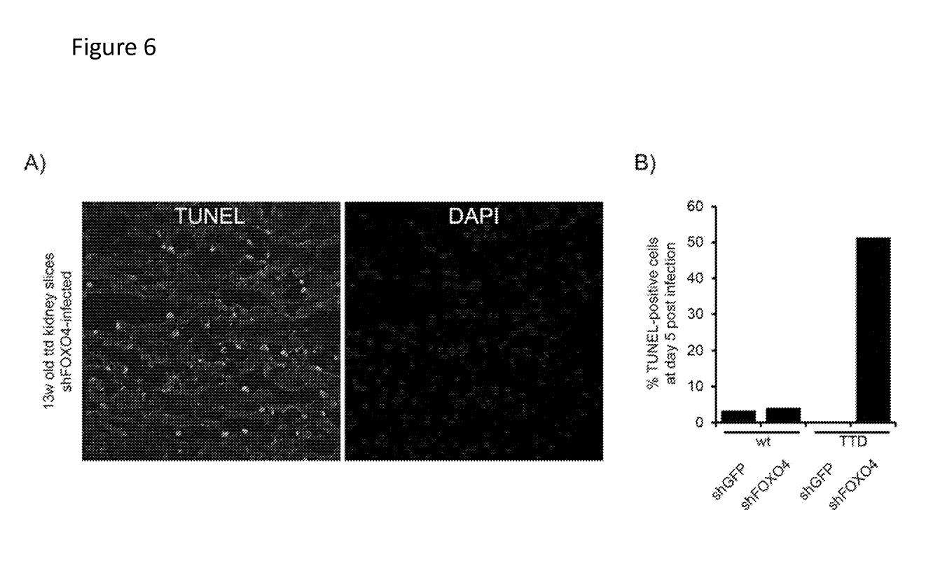

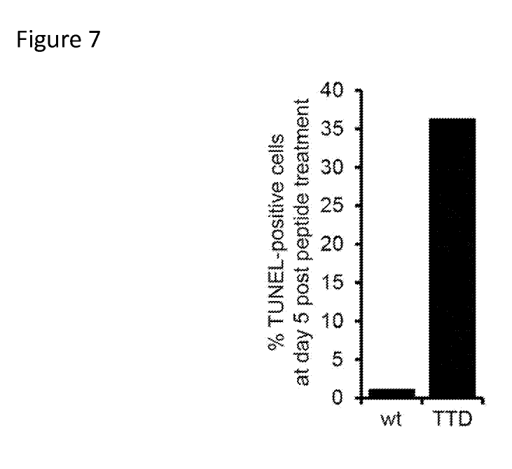

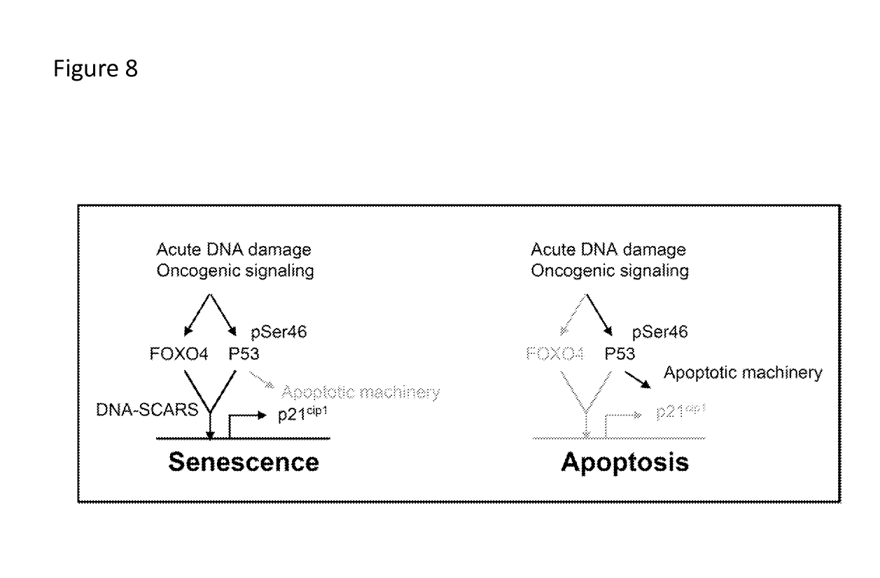

To investigate the effects of FOXO4 inhibition on age-associated senescence we isolated kidneys from 13 week old TTD mice and sliced them for culture ex vivo. Infection of TTD, but not wildtype, kidney slices with lentiviral particles containing FOXO4 knockdown constructs resulted in a strong apoptosis response in time (FIG. 6). A similar effect was observed when the TTD slices were exposed to the FOXO4-p53 blocking peptide mix (FIG. 7). Thus not only in stress-induced senescence, but also in age-associated senescence FOXO4 restrains apoptosis.

FIG. 9A: HEK293T cells were transiently transfected with PMT2-HA-FOXO4 in presence or absence of pEFm-BRAF^{V600E} using FuGENE according to the manufacturer's instructions (Roche). Two days after transfection cells were lysed in a 500 μl lysis buffer containing 20 mM Tris-HCl (pH 8.0), 1% TX-100, 0.5% NaDoC, 5 mM EDTA, 150 mM NaCl, 5 mM NaF, 1.25 mM NaVO₃, 10 μg/ml aprotenin and 10 μg/ml Leupeptide spun down for 10 min at 4° C. and processed for immunoprecipitation. In short, 50 μl 20% protein A coated sepharose beads (Roche) were incubated for 1 h at 4° C. with 1 μg of an antibody against endogenous p53 (D01, Santa Cruz Biotechnology) in 100 μl lysis buffer. The antibody-coupled beads were subsequently washed twice in 1 ml lysis buffer, spun down, incubated for 2 hours at 4° C. with 400 μl of the supernatant of the cellular lysate, washed three times with 1 ml lysis buffer and processed for SDS-PAGE. 20 μl of the remaining supernatant was loaded as an input control. Proteins on the western blot were detected using antibodies against FOXO4 (834), p53 (D01, Santa Cruz Biotechnology) and phosphoThr183/Tyr185-JNK (4668S, Cell signaling) as a marker for active BRAF^{V600E} signaling. Another experiment was conducted similarly as in A), except that the cells were incubated or left untreated for 24 h prior to lysis with 10 μM of the MEK-inhibitor U0126. On the western blot phosphoThr202/Tyr204-ERK (4370S, Cell signaling) was used as a marker of MEK activity. FIG. 9B: BRAF^{V600E}-mutated A375 malignant melanoma cells were infected as in FIG. 2a and selected for 3 days with 1 μg/ml Puromycin. Subsequently 2×10⁴ cells were plated in triplicate on glass coverslips in 24-well plate chambers. The next day the cells were incubated with the indicated concentrations of the BRAF^{V600E}-inhibitor PLX4032 in the presence of 20 μM of the general caspase-inhibitor Q-VD-OPH. After 5 days the cells were processed as in FIG. 2b for Cytochrome C staining and the percentage of cells with Cytochrome C release was quantified. FIG. 9C: 5×10⁴ A375 melanoma cells were plated in triplicate in 96-well plate chambers. The next day the cells were incubated with or without a mixture containing 25 μM DBD2 peptide (sequence: GRKKRRQRRRPPPRKGGSRRNAWGNQSYAELISQAIESAPEKRLTL) and 25 μM C-tem2 peptide (Sequence: GRKKRRQRRRPPPQDLDLDMYMENLECDMDNIISDLMDEGEGLDF) in the presence or absence of 0.5 μM PLX4032. After 6 days the a CellTiter 96® AQueous One Solution Cell Proliferation Assay (MTS) was performed according to the manufacturer's instructions (Promega) to determine the relative number of cells versus Mock treated A375 melanoma cells in presence or absence of the peptide mixture.

FIG. 6A: NRAS^{Q61L}-mutated D04 were infected as in FIG. 2a and selected for 3 days with 0.5 μg/ml Puromycin. 5×10⁵ cells were plated in triplicate in 6 cm dishes and left for 10 days after which cells were fixed 10 min at room temperature with MeOH, incubated for 1 min with 0.5% Crystal Violet in 25% MeOH and washed thoroughly with Demi-H₂O (Top panels). In parallel, at day 5 after plating brightfield images were taken to show morphological changes in the cells (Middle panels). Also in parallel, after puromycin selection 2×10⁵ cells were plated on coverslips and processed for Cytochrome C release staining as in FIG. 2B) and the number of cells with released Cytochrome C quantified (100 cells). FIG. 6B: 1.5×10⁵ D04 cells were plated in 24-well plates. The next day the cells were incubated with 50 μM DBD1-peptide peptide (sequence: GRKKRRQRRRPPSRRNAWGNQSYAELIS), 50 μM C-tem1 peptide (Sequence: GRKKRRQRRRPPLECDMDNIISDLMDEGEGLDF) or a mix of both. After 6 days of incubation total cell number was quantified and the fold increase in number calculated.

FIG. 11A: 2×10⁵ A375 melanoma cells were plated in 24-well plate chambers. The next day the cells were incubated with 50 μM DBD2-peptide or a mixture of 25 μM DBD2-peptide and 25 μM C-term peptide in the presence or absence of 2 μM Cisplatin. Four days later brightfield images were taken to show rounding up of cells, notably in the condition where Cisplatin was combined with the peptide mixture. FIG. 11B: Similar experiment as in FIG. 9C, except with a different cell line, MCF7 mammary carcinoma, and a different compound, 5′-FluoroUracyl. FIG. 11C: Similar experiment as in FIG. 9C, except with B16F10LUC cells, infected with a mouse-specific shFOXO4 (#5; sequence: 5′-CAAGTTCATCAAGGTTCACAA-3′) and human shFOXO4-2 as used in FIG. 2b in presence or absence of 10 μM Cisplatin.

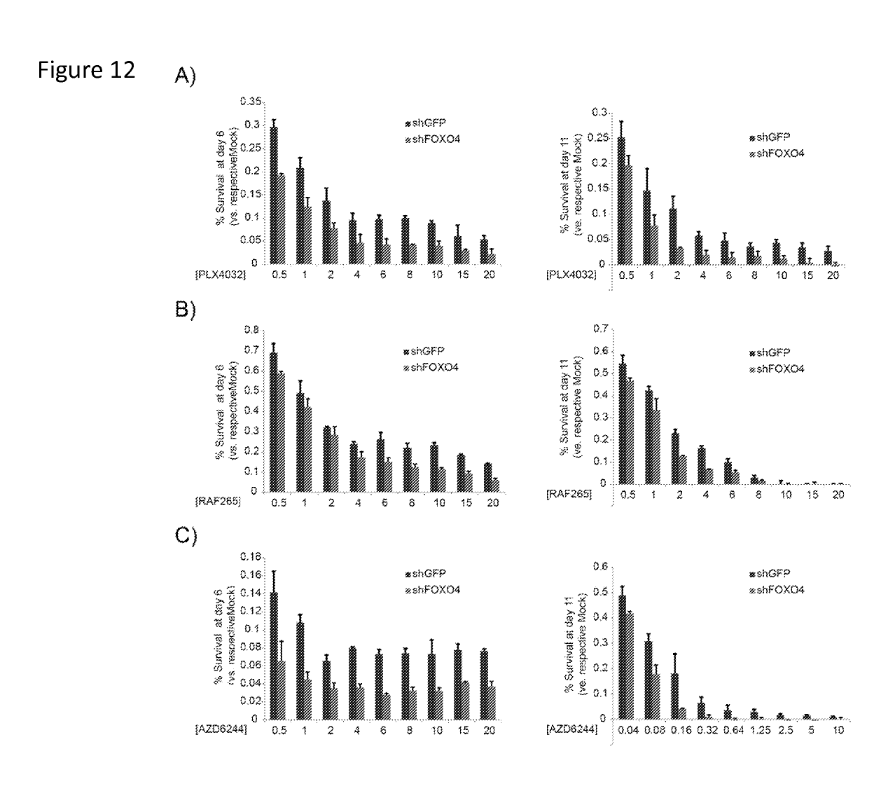

FIG. 12A: 2×10⁴ A375-shGFP or A375-shFOXO4-2 cells were plated in triplicate in 96-well plate chambers. The next day the cells were incubated with the indicated concentrations of PLX4032 or left untreated. After 6 days the a CellTiter 96® AQueous One Solution Cell Proliferation Assay (MTS) was performed according to the manufacturer's instructions (Promega) to determine the relative number of cells versus Mock treated A375-shGFP or A375-shFOXO4-2 melanoma cells (Left panel). Additionally, in a separate experiment the cells were refreshed at day 7 with the same dose of PLX4032 and left for another 4 days after which the same procedure was performed. FIG. 12B: Similar experiment as in A), except with the general RAF inhibitor RAF265. FIG. 12C: Similar experiment as in A), except with the MEK inhibitor AZD6244.

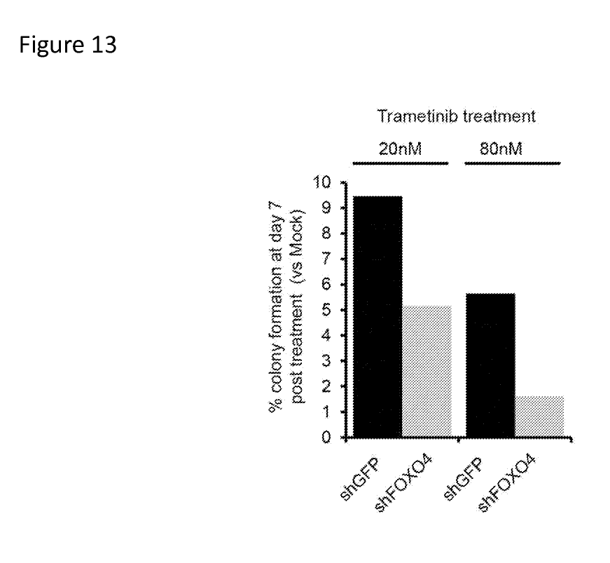

In addition to Vemurafenib, AZD6244 and Raf264 we also investigated the sensitivity of A375 melanoma cells to the MEK-inhibitor Trametinib. Concentrations as low as 80 nM resulted in a near-complete loss of colony forming potential of FOXO4-depleted, but not FOXO4-expressing, A375 melanoma cells (see FIG. 13). Given that recurrence of therapy-surviving cancer cells is the main cause of chemoresistance and lethality of patients, this result suggests that as adjuvant treatment FOXO4 inhibition could greatly benefit melanoma patients receiving Trametinib.

### Example 2

### Making FOXO4 Blocking Peptides

The amino acid sequence of human FOXO4 is shown as follows (SEQ ID NO:1):

MDPGNENSATEAAAIIDLDPDFEPQSRPRSCTWPLPRPEIANQPSEP
PEVEPDLGEKVHTEGRSEPILLPSRLPEPAGGPQPGILGAVTGPRKG
GSRRNAWGNQSYAELISQAIESAPEKRLTLAQIYEWMVRTVPYFKDK
GDSNSSAGWKNSIRHNLSLHSKFIKVHNEATGKSSWWMLNPEGGKSG
KAPRRRAASMDSSSKLLRGRSKAPKKKPSVLPAPPEGATPTSPVGHF
AKWSGSPCSRNREEADMWTTFRPRSSSNASSVSTRLSPLRPESEVLA
EEIPASVSSYAGGVPPTLNEGLELLDGLNLTSSHSLLSRSGLSGFSL
QHPGVTGPLHTYSSSLFSPAEGPLSAGEGCFSSSQALEALLTSDTPP
PPADVLMTQVDPILSQAPTLLLLGGLPSSSKLATGVGLCPKPLEAPG
PSSLVPTLSMIAPPPVMASAPIPKALGTPVLTPPTEAASQDRM [symbol]
NFEPDP

The DNA binding domain as defined in Greer, E. L et al. (Oncogene 24, 7410-7425 (2005)) is underlined. The peptides of which the sequence lies within this domain are called the DBD-peptides. Two amino acids (WG) which in the FOXO4 homolog FOXO3 show the largest NMR shift upon addition of recombinant p53 are bolded and underlined. The peptides of which then sequence is localized in the far c-terminus are called C-term peptides. This region contains the Transactivation domain (van der Horst, A. et al. Nat. Rev. Mol. Cell Biol. 8, 440-450 (2007)). The longer C-term peptide used in the studies is bolded and italicized in the sequence above.

The amino acid sequence for DNA binding domain of FOXO4 is designated as SEQ ID NO:2:

ILGAVTGPRKGGSRRNAWGNQSYAELISQAIESAPEKRLTLAQIYEW
MVRTVPYFKDKGDSNSSAGWKNSIRHNLSLHSKFIKVHNEATGKSSW
WMLNPEGGKSGKAPRRRAASMDSSS

The C-terminal portion of FOXO4 with sequence below is designated as SEQ ID NO:3:

PQDLDLDMYMENLECDMDNIISDLMDEGEGLDFNFEPDP

We were interested in blocking the interaction between p53 and FOXO4. We have shown that FOXO4 and p53 can physically interact, but the interaction domains are not characterized yet. By means of NMR the in vitro interaction of FOXO3a with p53 has been determined to occur at two independent sites (Wang, F. et al. J Mol Biol 384, 590-603 (2008)). Based on sequence homology between FOXO3a and FOXO4, we designed two peptides for each interaction domain that could potentially block the physical interaction of FOXO4 with p53.

The DBD-peptides (in the vicinity for the first p53 interaction domain in FOXO3a) are shown in FIG. 9A. The NRM data from this paper suggest that W157 and G158 in FOXO3a shift the most when p53 is added. The DNA binding domain (DBD) of FOXO3a has been characterized to be between aa141-266 which contains W157 and G158. Therefore we called the peptide that could potentially block the p53-FOXO interaction in this domain the DBD-peptides. We designed two peptides of different lengths (sequence from DBD1 underscored):

| | |
|---|---|
| DBD1: | SRRNAWGNQSYAELIS |
| DBD2: | PRKGGSRRNAWGNQSYAELISQAIESAPEKRLTL |

The C-Terminal peptides (in the vicinity for the second p53 interaction domain in FOXO3a) are shown in FIG. 9B. The homologous domain in FOXO4 is in the far C-terminus Therefore we called the peptide that could potentially block the p53-FOXO4 interaction in this domain the C-term peptides (sequence from C-term1 underscored).

| | |
|---|---|
| C-term1: | LECDMDNIISDLMDEGEGLDF |
| C-term2: | PQDLDLDMYMENLECDMDNIISDLMDEGEGLDF |

TAT-PP was added to these peptides to facilitate cellular uptake. In order to be able to facilitate entry of peptides into cells we used a system based on the TAT-protein from the Human Immunodeficiency Virus, which has been shown to be usable for that purpose in vitro and in vivo for other peptides (Bonny, C et al. Diabetes 50, 77-82 (2001); Kang, W. H. et al., J Neurotrauma 28, 1219-1228, doi:10.1089/neu.2011.1879 (2011)).

The TAT-PP sequence (TAT with two prolines added c-terminally for flexibility) that can be used for transfer peptides across the plasma-membrane has the following sequence: GRKKRRQRRRPP (Ruben, S. et al. J Virol 63, 1-8 (1989); Fawell, S. et al. Proc Natl Acad Sci USA 91, 664-668 (1994); Vives, E. et al. J Biol Chem 272, 16010-16017 (1997)).

The full peptides used in the studies have the following sequences:

FOXO4-p53 DBD1:
GRKKRRQRRRPPSRRNAWGNQSYAELIS
FOXO4-p53 DBD2:
GRKKRRQRRRPPPRKGGSRRNAWGNQSYAELISQAIESAPEKRLTL
FOXO4-p53 C-term1:
GRKKRRQRRRPPLECDMDNIISDLMDEGEGLDF
FOXO4-p53 C-term2:
GRKKRRQRRRPPPQDLDLDMYMENLECDMDNIISDLMDEGEGLDF

The combination of FOXO4-p53 DBD2 with FOXO4-p53 C-term1 appears to work best in sensitizing melanoma cells to chemotherapy.

### Example 3

### Studies Using Additional Chemotherapeutic Agents

We have shown that A375-shFOXO4 melanoma cells are more sensitive than A375-shGFP melanoma cells to not only PLX4032 (from Roche), but also to RAF265 (from Novartis) and AZD6244 (from Astra-Zeneca) and Cisplatin. How plain A375 cells respond to these drugs in the presence of a mixture of the DBD and C-term peptides is studied. The experimental design is as follows: 2×10⁴ A375 melanoma cells plated in triplicate in 96-well plate chambers. The next day the cells are incubated with or without a mixture containing the DBD2 peptide (sequence: GRKKRRQRRRPPPRKGGSRRNAWGNQSYAELISQAIESAPEKRLTL) and the C-tem2 peptide (Sequence: GRKKRRQRRRPPPQDLDLDMYMENLECDMDNIISDLMDEGEGLDF), or either peptide alone, in different concentrations in the presence or absence of PLX4032, RAF265, AZD6244 or Cisplatin. After 6 days the a CellTiter 96® AQueous One Solution Cell Proliferation Assay (MTS) is performed according to the manufacturer's instructions (Promega) to determine the relative number of cells versus Mock treated A375 melanoma cells in presence or absence of the peptide mixture.

### Example 4

### Studies Using NRAS-Mutated Melanoma Cells and PLX4032-Resistant BRAFV600E-Driven Melanoma Cells for Sensitivity to Chemotherapy by FOXO4 Interference

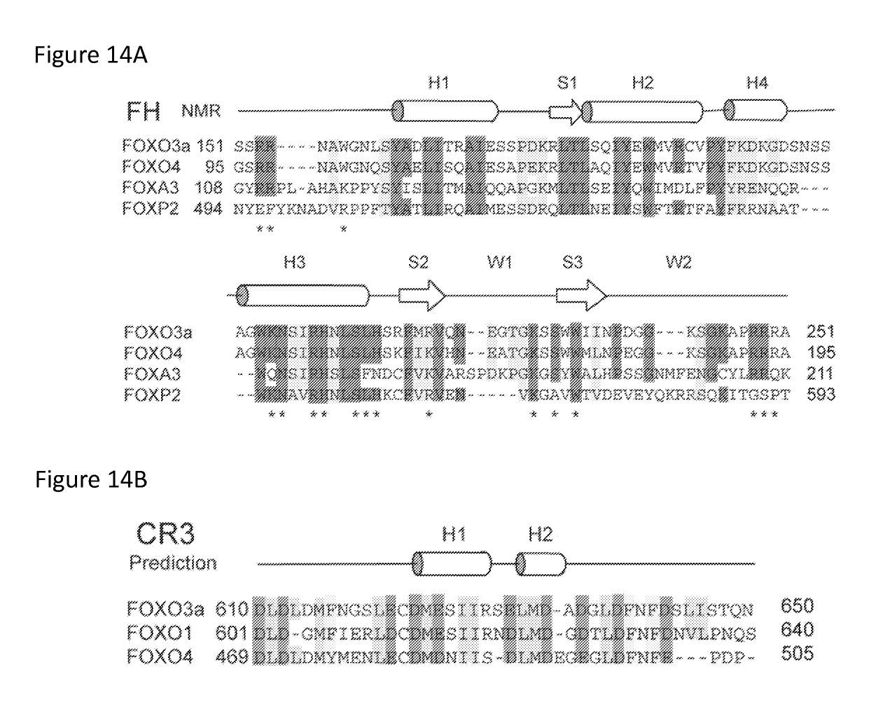

PLX4032-resistant A375 melanoma cells are generated through chronic treatment with high concentrations (10 μM) of PLX4032. PLX4032-resistant A375 cells are infected as in FIG. 14c or treated with peptides as described above under Example 3 and similarly tested for sensitivity to AZD6244, RAF265 and Cisplatin.

### Example 5

### Studies Using Additional Cancer Types

Tumor cells such as pancreatic cancer, ovarian cancer, glioblastoma, certain renal cancers, mammary carcinoma, and prostate carcinoma are infected as in FIG. 14c or treated with peptides as described above under Example 3 and similarly tested for sensitivity to AZD6244, 5′-FluoroUracyl, Doxorubicin and Mitoxantrone.

### Example 6

### Studying the Effects of FOXO4 Interference on the Sensitivity of Normal Cells Towards Chemotherapy

Primary human melanocytes did not appear to suffer from the FOXO4-p53 blocking peptides under basal conditions. Where tested, BRAF and MEK inhibitors affect normal cells with a much lesser efficiency than cancer cells, which provides potential for clinical use of these in combination with the FOXO4-p53 blocking peptides. Normal human IMR90 fibroblasts and primary human melanomacytes are plated under similar conditions as under Example 3 and treated with PLX4032, RAF265, AZD6244, Cisplatin, 5′-FluoroUracyl, Doxorubicin and Mitoxantrone.

### Example 7

### Studying the Effects of FOXO4 Interference on Tumor Growth in Xenograft and Metastasis Models In Vivo in Mice

For the xenograft study, we use A375 cells, which others (Kinders, R. J. et al. Clin Cancer Res 16, 5447-5457, (2010)) and we (not shown) have found to easily form tumors in mice and are responsive to BRAF^{V600E}-inhibition in vivo (Yang, H. et al. Cancer Res 70, 5518-5527, (2010)). For the metastasis study, we first make use of a mouse melanoma cell line, B16F10luc, which has great metastatic potential in the lung when injected intravenously (Kashani-Sabet, M. et al. Proc Natl Acad Sci USA 99, 3878-3883, (2002)). Since this line stably expresses the firefly luciferase reporter gene luminescence intensity is followed in time in live animals as a measure of lung metastases. Importantly, B16F10Luc incubated with the p53-FOXO4 blocking peptide mix are also sensitive to at least cisplatin (FIG. 11c). Additional chemotherapies are tested before conducting the in vivo experiments.

Nu/nu mice are injected sub cutaneous with 5×10⁶ A375Luc (shGFP and shFOXO4). After one week of tumor growth drug treatment is initiated. For AZD6244, 25 mg/kg of drug is administered twice daily through oral gavage for 10 days. 10 animals are used per group. Tumor size is monitored weekly by luminescence using Caliperls IVIS® detection system. Experiment is ended by euthanizing the mouse when tumor reached a size of 2 cubic centimeters or more.

c57b1/6 albino mice are used for injection of B 16F10LUC mouse melanoma cells. 5×10⁵ cells are injected into mice tail vein. PLX4032, RAF265, AZD6244 and Cisplatin under similar conditions as described under Example 3. Drug treatments are initiated 24 hours after cells injection. Luminescence is evaluated twice a week. Experiment end with animal death from metastasis. Autopsy is then performed to evaluate metastasis.

### Example 8

### Studying the Effects of FOXO4 Inhibition on Ageing Phenotypes

This example examines the effects of FOXO4 inhibition on aging phenotypes in an appropriate progeroid mouse model such as shown by (Baker, D. J. et al. Nature 479, 232-236, 2011) or in XPD mutant (Andressoo, J. O. et al. Mol Cell Biol 29, 1276-1290, 2009) or ERCC1 mutant (Weeda, G. et al. Curr. Biol. 7, 427-439 (1997); de Waard, M. C. et al. Acta Neuropathol 120, 461-475, (2010)) mouse models.

IMR90 cells are infected with lentiviral particles containing shGFP or shFOXO4-1 and -2 as in FIG. 2A. Following selection with 0.5 μg/ml puromycin 2×10⁴ cells are plated in triplicate in 96-well chambers. The next day the cells are left untreated or are irradiated with 10Gy XRAY. After 6 days the a CellTiter 96® AQueous One Solution Cell Proliferation Assay (MTS) is performed according to the manufacturer's instructions (Promega) to determine the relative number of cells versus Mock treated IMR90 cells.

A similar experiment is performed on non-infected IMR90 cells in presence or absence of the peptide mixture. 2×10⁴ IMR90 cells are plated in triplicate in 96-well plate chambers. The next day the cells are incubated with or without a mixture containing the DBD2 peptide (sequence: GRKKRRQRRRPPPRKGGSRRNAWGNQSYAELISQAIESAPEKRLTL) and the C-tem2 peptide (Sequence: GRKKRRQRRRPPPQDLDLDMYMENLECDMDNIISDLMDEGEGLDF), or either peptide alone, and irradiated with 10Gy XRAY or left untreated. After 6 days the a CellTiter 96® AQueous One Solution Cell Proliferation Assay (MTS) is performed according to the manufacturer's instructions (Promega) to determine the relative number of cells versus Mock treated IMR90 cells.

### Example 9

### Delivery of FOXO4-Inhibitory Agents

We have described the inhibition of FOXO4 for sensitization of senescent and cancer cells towards apoptosis by two methods: Stable (lentiviral) shRNA-based knockdown (RNAi) and FOXO4-p53 blocking peptides. We further use additional means to inhibit FOXO4 expression or activity.

Delivery of FOXO4-p53 blocking peptides by liposomes is used. Thus far, we made use of an approved method of peptide delivery using the TAT sequence of HIV, which facilitates peptide entry into cells. The use of this method has been demonstrated in vivo in numerous disease models. The peptides used in this method may become quite long and therefore more expensive. Delivery by liposomes would allow shorter peptides to be delivered. Additionally, the stability of peptides in vivo is relatively short. The use of liposomes may improve stability prior to delivery to the desired cells.

Additional methods that could facilitate (A) knockdown of FOXO4 in vivo or (B) deliver FOXO4 inhibitory peptides in vivo are also used.

A) Delivery of FOXO4-targeting siRNA using cell permeable peptides (such as, but not limited to, CADY, MPG and Pepl). Cell Permeable peptides have been reported to form stable complexes with siRNA and facilitate their uptake in cells in vivo (Morris, M. C. et al. Biol Cell 100, 201-217, (2008)). These include, but are not limited to, the primary amphipatic peptides MPG (GALFLGFLGAAGSTMGAWSQPKKKRKV) and Pep-1 (KETWWETWWTEWSQPKKKRKV), either in their primary or modified forms to enhance stability (Morris, M. C. et al. Biol Cell 100, 201-217, (2008)) and the secondary amphipathic peptide CADY (Ac-GLWRALWRLLRSLWRLLWRA-Cya) (Konate, K. et al. Biochemistry 49, 3393-3402, (2010)). Binding of the siRNA typically induces a conformational change in the peptides resulting in enhanced stabilization of the peptide-siRNA complex. In order to facilitate knockdown of FOXO4 in vivo we test whether CPP/anti-FOXO4 siRNA complexes can be used, thereby potentially targeting senescent and cancer cells for apoptosis.

B) Delivery of FOXO4-p53 inhibitory peptides using fusion with permeable peptides. Next to the TAT-sequence we used to deliver FOXO4-blocking peptides, also Pepl has been shown to be able to deliver peptides in vivo—be it not through fusion with the peptide, but complexation. Also, recently octa-arginine (R(8))-peptide has been shown to, when indeed fused to a cargo peptide, be able to be taken up in cells and have a biological function (Fricke, T. et al. Bioconjug Chem 22, 1763-1767, (2011)).

### Example 10

### Assessing the Effects of shRNA-Mediated Downregulation of FOXO4 Activity on Cancer Relapse in Combination Therapy

Various cancers relapse because some cells survive chemotherapy treatment. Decreasing FOXO4 activity sensitizes various cancer cells to chemotherapy, and thus prevents relapses of cancer in vivo and improve survival. The effects of decreasing FOXO4 activity on cancer relapse is assessed in a tumor allograft or tumor xenograft model in mice.

Melanoma

Three groups of melanoma cancer cell lines (derived from e.g., BRAF^{V600E}-mutated human melanoma cell line A375, NRAS^{Q61K}-mutated human melanoma cell line D04, or mouse melanoma cell line B-16F10) are prepared for injection into mice. The first cell line is a melanoma cancer cell line that has been stably transduced with a virus encoding an shRNA against FOXO4, such as described above. The second cell line is a non-infected melanoma cell line. The third cell line is a melanoma cell line infected with a virus expressing a random scramble shRNA.

Each of the three melanoma cell lines described above is injected sub-cutaneously into a different group of mice. When cell lines of human origin are used, the injection is performed in athymic nude mice. When cell lines of mouse origin are used, the injection is performed in syngeneic mice. Tumor formation is assessed every other day by palpation and measured with a caliper. When the tumor is detected, chemotherapy treatment (e.g., inhibitors of the RAF-MEK-ERK kinase pathway, such as Trametinib or RAF265) is initiated in half of the mice in each group, and vehicle control treatment is initiated in the other half. Mice injected with non-infected cells melanoma cells are infected with the virus encoding an shRNA against FOXO4 in vivo prior to or at the time chemotherapy (or vehicle control) treatment. Chemotherapy treatment is stopped when tumor volume shrinks or ceases to grow for about a week. Tumor size is monitored for two months to evaluate relapse. Control animals are sacrificed when the tumor reaches a volume of more than 2 cm³.

Breast Cancer

Three groups of breast cancer cell lines (derived from, e.g., human mammary carcinoma cell lines MCF7, T47D, or MDA-MB-231, or mouse mammary carcinoma cell line 4T1) are prepared for injection into mice. The first cell line is a breast cancer cell line that has been stably transduced with a virus encoding an shRNA against FOXO4, such as described above. The second cell line is a non-infected breast cancer cell line. The third cell line is a breast cancer cell line infected with a virus expressing a random scramble shRNA.

Each of the three breast cancer cell lines described above is injected sub-cutaneously or into the mammary fat pads of a different group of mice. When cell lines of human origin are used, the injection is performed in athymic nude mice. When cell lines of mouse origin are used, the injection is performed in syngeneic mice. Tumor formation is assessed every other day by palpation and measured with a caliper. When the tumor is detected, chemotherapy treatment (e.g., 5-fluorouracil, the alkylating agent 4-hydroperoxycyclophosphamide, and doxorubicin) is initiated in half of the mice in each group, and vehicle control treatment is initiated in the other half. Mice injected with non-infected cells breast cancer cells are infected with the virus encoding an shRNA against FOXO4 in vivo prior to or at the time of chemotherapy (or vehicle control) treatment. Chemotherapy treatment is stopped when tumor volume shrinks or ceases to grow for about a week. Tumor size is monitored for two months to evaluate relapse. Control animals are sacrificed when the tumor reaches a volume of more than 2 cm³.

Prostate Cancer

Three groups of prostate cancer cell lines (derived from, e.g., human prostate cancer cell line PC3 or mouse prostate cancer cell lines) are prepared for injection into mice. The first cell line is a prostate cancer cell line that has been stably transduced with a virus encoding an shRNA against FOXO4, such as described above. The second cell line is a non-infected prostate cancer cell line. The third cell line is a prostate cancer cell line infected with a virus expressing a random scramble shRNA.

Each of the three prostate cancer cell lines described above is injected sub-cutaneously into a different group of mice. When cell lines of human origin are used, the injection is performed in athymic nude mice. When cell lines of mouse origin are used, the injection is performed in syngeneic mice. Tumor formation is assessed every other day by palpation and measured with a caliper. When the tumor is detected, chemotherapy treatment (e.g., Paclitaxel, doxorubicin and mitoxantrone) is initiated in half of the mice in each group, and vehicle control treatment is initiated in the other half. Mice injected with non-infected cells prostate cancer cells are infected with the virus encoding an shRNA against FOXO4 in vivo prior to or at the time of chemotherapy (or vehicle control) treatment. Chemotherapy treatment is stopped when tumor volume shrinks or ceases to grow for about a week. Tumor size is monitored for two months to evaluate relapse. Control animals are sacrificed when the tumor reaches a volume of more than 2 cm³.

Colorectal Cancer (CRC)

Three groups of colorectal cancer (CRC) cell lines (derived from, e.g., human colorectal cancer cell lines HCT116, SW480, or DLD1 or mouse colorectal cancer cell line Colon-26) are prepared for injection into mice. The first cell line is a CRC cell line that has been stably transduced with a virus encoding an shRNA against FOXO4, such as described above. The second cell line is a non-infected CRC cell line. The third cell line is a CRC cell line infected with a virus expressing a random scramble shRNA.

Each of the three CRC cell lines described above is injected sub-cutaneously into a different group of mice. When cell lines of human origin are used, the injection is performed in athymic nude mice. When cell lines of mouse origin are used, the injection is performed in syngeneic mice. Tumor formation is assessed every other day by palpation and measured with a caliper. When the tumor is detected, chemotherapy treatment (e.g., 5-fluorouracil and cisplatin) is initiated in half of the mice in each group, and vehicle control treatment is initiated in the other half. Mice injected with non-infected cells CRC cells are infected with the virus encoding an shRNA against FOXO4 in vivo prior to or at the time of chemotherapy (or vehicle control) treatment. Chemotherapy treatment is stopped when tumor volume shrinks or ceases to grow for about a week. Tumor size is monitored for two months to evaluate relapse. Control animals are sacrificed when the tumor reaches a volume of more than 2 cm³.

### Example 11

### Assessing the Effects of Peptide-Mediated Downregulation of FOXO4 Activity on Cancer Relapse in Combination Therapy

Melanoma

Melanoma cancer cells (e.g., BRAF^{V600E}-mutated human melanoma cell line A375, NRAS^{Q61K}-mutated human melanoma cell line D04, or mouse melanoma cell line B-16F10) are injected sub-cutaneously into mice. When cell lines of human origin are used, the injection is performed in athymic nude mice. When cell lines of mouse origin are used, the injection is performed in syngeneic mice.

Tumor formation is assessed every other day by palpation and measured with a caliper. When the tumor is detected, chemotherapy treatment (e.g., inhibitors of the RAF-MEK-ERK kinase pathway, such as Trametinib or RAF265) is initiated in half of the mice in each group, and vehicle control treatment is initiated in the other half.

Half of the mice receiving chemotherapy also receive a peptide preventing p53-FOXO4 interaction, such as those described above, and the other half of the mice receiving chemotherapy receive vehicle. Similarly, half the mice that are not receiving chemotherapy receive the peptide preventing p53-FOXO4 interaction, such as those described above, and the other half of the mice not receiving chemotherapy receive vehicle. The peptide or vehicle is injected intra-tumor. When tumor volume shrinks or ceases to grow for a week, treatment is stopped. Tumor size is monitored for two months in all four groups of mice (i.e., chemotherapy+peptide, chemotherapy+no peptide vehicle, no chemotherapy vehicle+peptide, and no chemotherapy vehicle+no peptide vehicle) to evaluate relapse.

A similar experiment to that described above is performed in which the peptide or vehicle control is introduced intravenously.

Breast Cancer

Breast cancer cells (e.g., human mammary carcinoma cell lines MCF7, T47D, or MDA-MB-231, or mouse mammary carcinoma cell line 4T1) are injected sub-cutaneously or into the mammary fat pad of mice. When cell lines of human origin are used, the injection is performed in athymic nude mice. When cell lines of mouse origin are used, the injection is performed in syngeneic mice.

Tumor formation is assessed every other day by palpation and measured with a caliper. When the tumor is detected, chemotherapy treatment (e.g., 5-fluorouracil, the alkylating agent 4-hydroperoxycyclophosphamide, and doxorubicin) is initiated in half of the mice in each group, and vehicle control treatment is initiated in the other half.

Half of the mice receiving chemotherapy also receive a peptide preventing p53-FOXO4 interaction, such as those described above, and the other half of the mice receiving chemotherapy receive vehicle. Similarly, half the mice that are not receiving chemotherapy receive the peptide preventing p53-FOXO4 interaction, such as those described above, and the other half of the mice not receiving chemotherapy receive vehicle. The peptide or vehicle is injected intra-tumor. When tumor volume shrinks or ceases to grow for a week, treatment is stopped. Tumor size is monitored for two months in all four groups of mice (i.e., chemotherapy+peptide, chemotherapy+no peptide vehicle, no chemotherapy vehicle+peptide, and no chemotherapy vehicle+no peptide vehicle) to evaluate relapse.

A similar experiment to that described above is performed in which the peptide or vehicle control is introduced intravenously.

Prostate Cancer

Prostate cancer cells (e.g., human prostate cancer cell line PC3 or mouse prostate cancer cell lines) are injected sub-cutaneously into mice. When cell lines of human origin are used, the injection is performed in athymic nude mice. When cell lines of mouse origin are used, the injection is performed in syngeneic mice.

Tumor formation is assessed every other day by palpation and measured with a caliper. When the tumor is detected, chemotherapy treatment (e.g., Paclitaxel, doxorubicin and mitoxantrone) is initiated in half of the mice in each group, and vehicle control treatment is initiated in the other half. Half of the mice receiving chemotherapy also receive a peptide preventing p53-FOXO4 interaction, such as those described above, and the other half of the mice receiving chemotherapy receive vehicle. Similarly, half the mice that are not receiving chemotherapy receive the peptide preventing p53-FOXO4 interaction, such as those described above, and the other half of the mice not receiving chemotherapy receive vehicle. The peptide or vehicle is injected intra-tumor. When tumor volume shrinks or ceases to grow for a week, treatment is stopped. Tumor size is monitored for two months in all four groups of mice (i.e., chemotherapy+peptide, chemotherapy+no peptide vehicle, no chemotherapy vehicle+peptide, and no chemotherapy vehicle+no peptide vehicle) to evaluate relapse.

A similar experiment to that described above is performed in which the peptide or vehicle control is introduced intravenously.

Colorectal Cancer (CRC)

CRC cells (e.g., human colorectal cancer cell lines HCT116, SW480, or DLD1 or mouse colorectal cancer cell line Colon-26) are injected sub-cutaneously into mice. When cell lines of human origin are used, the injection is performed in athymic nude mice. When cell lines of mouse origin are used, the injection is performed in syngeneic mice.

Tumor formation is assessed every other day by palpation and measured with a caliper. When the tumor is detected, chemotherapy treatment (e.g., 5-fluorouracil and cisplatin) is initiated in half of the mice in each group, and vehicle control treatment is initiated in the other half. Half of the mice receiving chemotherapy also receive a peptide preventing p53-FOXO4 interaction, such as those described above, and the other half of the mice receiving chemotherapy receive vehicle. Similarly, half the mice that are not receiving chemotherapy receive the peptide preventing p53-FOXO4 interaction, such as those described above, and the other half of the mice not receiving chemotherapy receive vehicle. The peptide or vehicle is injected intra-tumor. When tumor volume shrinks or ceases to grow for a week, treatment is stopped. Tumor size is monitored for two months in all four groups of mice (i.e., chemotherapy+peptide, chemotherapy+no peptide vehicle, no chemotherapy vehicle+peptide, and no chemotherapy vehicle+no peptide vehicle) to evaluate relapse.

A similar experiment to that described above is performed in which the peptide or vehicle control is introduced intravenously.

### Example 12

### Assessing the Effects of shRNA-Mediated Decrease in FOXO4 Activity on Survival of Metastatic Cancer in Combination Therapy

Melanoma

Two groups of melanoma cancer cell lines (derived from e.g., BRAF^{V600E}-mutated human melanoma cell line A375, NRAS^{Q61K}-mutated human melanoma cell line D04, or mouse melanoma cell line B-16F10) are constructed. The first cell line is a melanoma cell line transduced with a virus encoding an shRNA against FOXO4. The second cell line is a melanoma cell line transduced with a virus expressing a random scramble shRNA.

Each of the two melanoma cell lines is introduced into a different group of mice via tail vein injection or intracardiac injection to establish metastasis. When cell lines of human origin are used, the injection is performed in athymic nude mice. When cell lines of mouse origin are used, the injection is performed in syngeneic mice.

After one week, half of the mice in each group are treated with chemotherapeutic agent (e.g., inhibitors of the RAF-MEK-ERK kinase pathway, such as Trametinib or RAF265), and the other half is given vehicle control. Survival is monitored.

Breast Cancer

Two groups of breast cancer cell lines (derived from e.g., human mammary carcinoma cell lines MCF7, T47D, or MDA-MB-231, or mouse mammary carcinoma cell line 4T1) are constructed. The first cell line is a breast cell line transduced with a virus encoding an shRNA against FOXO4. The second cell line is a breast cancer cell line transduced with a virus expressing a random scramble shRNA.

Each of the two breast cancer cell lines is introduced into a different group of mice via tail vein injection or intracardiac injection to establish metastasis. When cell lines of human origin are used, the injection is performed in athymic nude mice. When cell lines of mouse origin are used, the injection is performed in syngeneic mice.

After one week, half of the mice in each group are treated with chemotherapeutic agent (e.g., 5-fluorouracil, they alkylating agent 4-hydroperoxycyclophosphamide, and doxorubicin), and the other half is given vehicle control. Survival is monitored.

Prostate Cancer

Two groups of prostate cancer cell lines (derived from, e.g., human prostate cancer cell line PC3 or mouse prostate cancer cell lines) are constructed. The first cell line is a prostate cancer cell line transduced with a virus encoding an shRNA against FOXO4. The second cell line is a prostate cancer cell line transduced with a virus expressing a random scramble shRNA.

Each of the two prostate cancer cell lines is introduced into a different group of mice via tail vein injection or intracardiac injection to establish metastasis. When cell lines of human origin are used, the injection is performed in athymic nude mice. When cell lines of mouse origin are used, the injection is performed in syngeneic mice.

After one week, half of the mice in each group are treated with chemotherapeutic agent (e.g., Paclitaxel, doxorubicin and mitoxantrone), and the other half is given vehicle control. Survival is monitored.

Colorectal Cancer (CRC)

Two groups of CRC cell lines (derived from, e.g., human colorectal cancer cell lines HCT116, SW480, or DLD1 or mouse colorectal cancer cell line Colon-26) are constructed. The first cell line is a CRC cell line transduced with a virus encoding an shRNA against FOXO4. The second cell line is a CRC cell line transduced with a virus expressing a random scramble shRNA.

Each of the two CRC cell lines is introduced into a different group of mice via tail vein injection or intracardiac injection to establish metastasis. When cell lines of human origin are used, the injection is performed in athymic nude mice. When cell lines of mouse origin are used, the injection is performed in syngeneic mice.

After one week, half of the mice in each group are treated with chemotherapeutic agent (e.g., Paclitaxel, doxorubicin and mitoxantrone), and the other half is given vehicle control. Survival is monitored.

### Example 13

### Assessing the Effects of Peptide-Mediated Decrease in FOXO4 Activity on Survival of Metastatic Cancer in Combination Therapy

Melanoma

Melanoma cancer cells (e.g., BRAF^{V600E}-mutated human melanoma cell line A375, NRAS^{Q61K}-mutated human melanoma cell line D04, or mouse melanoma cell line B-16F10) are injected into the circulatory system of mice via intracardiac injection or via tail vein injection. When cell lines of human origin are used, the injection is performed in athymic nude mice. When cell lines of mouse origin are used, the injection is performed in syngeneic mice.

After one week chemotherapy treatment (e.g., inhibitors of the RAF-MEK-ERK kinase pathway, such as Trametinib or RAF265) is initiated in half of the mice in each group, and vehicle control treatment is initiated in the other half. Half of the mice receiving chemotherapy also receive a peptide preventing p53-FOXO4 interaction, such as those described above, and the other half of the mice receiving chemotherapy receive vehicle. Similarly, half the mice that are not receiving chemotherapy receive the peptide preventing p53-FOXO4 interaction, such as those described above, and the other half of the mice not receiving chemotherapy receive vehicle. The peptide or vehicle is injected intra-tumor. Survival of all four groups of mice (i.e., chemotherapy+peptide, chemotherapy+no peptide vehicle, no chemotherapy vehicle+peptide, and no chemotherapy vehicle+no peptide vehicle) is monitored.

Breast Cancer

Breast cancer cells (e.g., human mammary carcinoma cell lines MCF7, T47D, or MDA-MB-231, or mouse mammary carcinoma cell line 4T1) are injected into the circulatory system of mice via intracardiac injection or via tail vein injection. When cell lines of human origin are used, the injection is performed in athymic nude mice. When cell lines of mouse origin are used, the injection is performed in syngeneic mice.

After one week chemotherapy treatment (e.g., 5-fluorouracil, the alkylating agent 4-hydroperoxycyclophosphamide, and doxorubicin) is initiated in half of the mice in each group, and vehicle control treatment is initiated in the other half. Half of the mice receiving chemotherapy also receive a peptide preventing p53-FOXO4 interaction, such as those described above, and the other half of the mice receiving chemotherapy receive vehicle. Similarly, half the mice that are not receiving chemotherapy receive the peptide preventing p53-FOXO4 interaction, such as those described above, and the other half of the mice not receiving chemotherapy receive vehicle. The peptide or vehicle is injected intra-tumor. Survival of all four groups of mice (i.e., chemotherapy+peptide, chemotherapy+no peptide vehicle, no chemotherapy vehicle+peptide, and no chemotherapy vehicle+no peptide vehicle) is monitored.

Prostate Cancer

Prostate cancer cells (e.g., human prostate cancer cell line PC3 or mouse prostate cancer cell lines) are injected into the circulatory system of mice via intracardiac injection or via tail vein injection. When cell lines of human origin are used, the injection is performed in athymic nude mice. When cell lines of mouse origin are used, the injection is performed in syngeneic mice.

After one week chemotherapy treatment (e.g., Paclitaxel, doxorubicin, and mitoxantrone) is initiated in half of the mice in each group, and vehicle control treatment is initiated in the other half. Half of the mice receiving chemotherapy also receive a peptide preventing p53-FOXO4 interaction, such as those described above, and the other half of the mice receiving chemotherapy receive vehicle. Similarly, half the mice that are not receiving chemotherapy receive the peptide preventing p53-FOXO4 interaction, such as those described above, and the other half of the mice not receiving chemotherapy receive vehicle. The peptide or vehicle is injected intra-tumor. Survival of all four groups of mice (i.e., chemotherapy+peptide, chemotherapy+no peptide vehicle, no chemotherapy vehicle+peptide, and no chemotherapy vehicle+no peptide vehicle) is monitored.

Colorectal Cancer (CRC)

CRC cells (e.g., human colorectal cancer cell lines HCT116, SW480, or DLD1 or mouse colorectal cancer cell line Colon-26) are injected into the circulatory system of mice via intracardiac injection or via tail vein injection. When cell lines of human origin are used, the injection is performed in athymic nude mice. When cell lines of mouse origin are used, the injection is performed in syngeneic mice.

After one week chemotherapy treatment (e.g., 5-fluorouracil and cisplatin) is initiated in half of the mice in each group, and vehicle control treatment is initiated in the other half. Half of the mice receiving chemotherapy also receive a peptide preventing p53-FOXO4 interaction, such as those described above, and the other half of the mice receiving chemotherapy receive vehicle. Similarly, half the mice that are not receiving chemotherapy receive the peptide preventing p53-FOXO4 interaction, such as those described above, and the other half of the mice not receiving chemotherapy receive vehicle. The peptide or vehicle is injected intra-tumor. Survival of all four groups of mice (i.e., chemotherapy+peptide, chemotherapy+no peptide vehicle, no chemotherapy vehicle+peptide, and no chemotherapy vehicle+no peptide vehicle) is monitored.

### Example 14

### Assessing the Effects of Decreased FOXO4 Activity on the Symptoms of Vascular Dysfunction and Atherosclerosis

Patients with atherosclerosis exhibit elevated presence of cell senescence in the blood and vessel walls (Gorenne et al. (2006) “Vascular smooth muscle cell senescence in atherosclerosis.” Cardiovasc Res 72: 9-17). Cell senescence has been shown to contribute to atherosclerosis (Minamino, et al. (2007) “Vascular cell senescence: contribution to atherosclerosis.” Circ Res 100: 15-26).

Trichothiodystrophy (TTD) is a rare, autosomal recessive nucleotide excision repair (NER) disorder caused by mutations in components of the dual functional NER/basal transcription factor TFIIH. TTD mice, which carry a patient-based point mutation in the Xpd gene, exhibit signs of premature aging and accelerated onset of senescence, and thus exhibit symptoms that resemble many features of the human syndrome. FOXO4 inhibition induces apoptosis in at least some tissues of TTD mice that were found to show elevated vascular dysfunction (Durik et al. (2012) “Nucleotide excision DNA repair is associated with age-related vascular dysfunction.” Circulation 126: 468-478).

To assess the role of FOXO4 activity in vascular dysfunction and atherosclerosis, a first group of TTD mice is transduced with FOXO4 shRNA viruses, such as those described above, and a second group of TTD mice is treated with FOXO4-p53 blocking peptides, such as those described above. A third group of TTD mice receives neither virus nor peptide. Symptoms of vascular dysfunction and atherosclerosis are measured in these three groups of mice and in control groups of syngeneic mice that do not carry the TTD mutation by laser Doppler perfusion imaging of reactive hyperemia after transient blood flow interruption as described (Durik et al. (2012) “Nucleotide-excision DNA repair is associated with age-related vascular dysfunction.” Circulation 126: 468-478). Post mortem, mice from each test group and from the control group are analyzed for the presence of senescent cells by SA-β-Gal staining.

### Example 15

### Assessing the Effects of Decreased FOXO4 Activity on the Symptoms of Diseases Associated with Cataracts

Cataracts are ocular spots that cloud the lens inside the eye, and the most common cause of cataract formation is aging (Truscott (2005). “Age-related nuclear cataract-oxidation is the key.” Exp Eye Res 80: 709-725). Cataracts have been associated with cellular senescence. BubR1^{H/H} mice are known to develop progeroid phenotypes, such as cataracts, at an accelerated pace due to earlier onset of senescence (Baker et al. (2004) “BubR1 insufficiency causes early onset of aging-associated phenotypes and infertility in mice.” Nature Gen 36: 744-749). Removal of senescent cells in a BubR1^{H/H} mouse model delays cataract formation (Baker et al. (2011) “Clearance of p16Ink4a-positive senescent cells delays ageing-associated disorders.” Nature 479: 232-236).

To assess the role of FOXO4 in cataract formation, a first group of BubR1^{H/H} mice is transduced with FOXO4 shRNA viruses, such as those described above, and a second group of BubR1^{H/H} mice is treated with FOXO4-p53 blocking peptides, such as those described above. A third group of BubR1^{H/H} mice receives neither virus nor peptide. Cataract formation is visually measured in time in each test group of mice and in control groups of syngeneic mice that do not carry the BubR1^{H/H} mutation. Post mortem, mice from each test group and control group are analyzed for the presence of senescent cells by SA-β-Gal staining.

### Example 16

### Assessing the Effects Decreased FOXO4 Activity on the Symptoms of Chronic Obstructive Pulmonary Disease (COPD) and Emphysema

Patients with chronic obstructive pulmonary disease (COPD) and emphysema exhibit elevated presence of cell senescence in the lungs (Tsuji et al. (2010) “Alveolar cell senescence exacerbates pulmonary inflammation in patients with chronic obstructive pulmonary disease.” Respiration 80: 59-70), suggesting a relationship between senescent cell accumulation and lung function disorder.

To assess the role of FOXO4 in emphysema or COPD, pulmonary disease is induced in mice by chronic exposure to cigarette smoke for six months, as described (Grumelli et al. (2011) “CD46 protects against chronic obstructive pulmonary disease.” PLoS One. 6: e18785). A first group of mice with induced pulmonary disease is transduced with FOXO4 shRNA viruses, such as those described above, a second group of mice with induced pulmonary disease is treated with FOXO4-p53 blocking peptides, such as those described above, and a third group of mice with induced pulmonary disease receives neither virus nor or peptide. Control groups include mice in which pulmonary disease was not induced. All mice receive rapamycin treatment, which has been shown to decrease airway remodeling and hyperreactivity (Kramer et al. (2011) “Rapamycin decreases airway remodeling and hyperreactivity in a transgenic model of non-inflammatory lung disease.” J Appl Physiol 111: 1760-1767). Two weeks following the beginning of rapamycin treatment, mice in from each test group and control group are subjected to an exercise test using a treadmill as described (Lüthje et al. (2009) “Exercise intolerance and systemic manifestations of pulmonary emphysema in a mouse model.” Respir Res. 10: 7). The mice from the test groups and control groups are then euthanized, and their lung tissue is prepared for pathology and assessed for senescence by SA-BGal staining and p16 expression, markers of the senescence-associated secretory phenotype (SASP) such as 1IL-1, IL-6, CXCL-1 and MMP3, and immune cell infiltration.

### Example 17

### Assessing the Effect of Decreased FOXO4 Activity on Diabetic Wound Healing

Patients with diabetic venous ulcer exhibit elevated presence of cellular senescence at sites of chronic wounds (Stanley et al. (2001) “Senescence and the healing rates of venous ulcers.” J Vasc Surg 33: 1206-1211). Chronic inflammation is observed at sites of chronic wound (Goren et al. (2006) “Severely impaired insulin signaling in chronic wounds of diabetic ob/ob mice: a potential role of tumor necrosis factor-alpha.” Am J. Pathol. 7 168: 65-77; Seitz et al. (2010) “Wound healing in mice with high-fat diet- or ob gene-induced diabetes-obesity syndromes: a comparative study.” Exp Diabetes Res. 2010: 476969), suggesting a role for the proinflammatory cytokine phenotype of senescent cells in the pathology.

The ob/ob mouse is a mutant mouse that eats excessively and becomes profoundly obese, and it is used as an animal model of type II diabetes. A first group of ob/ob mice is transduced with FOXO4 shRNA viruses, such as those described above, and a second group of ob/ob mice is treated with FOXO4-p53 blocking peptides, such as those described above. A third group of ob/ob mice receives neither virus nor peptide. Control groups include syngeneic mice that are not ob/ob. The shRNA transduction or peptide treatment is performed 14 days before wound induction.

Mice in all test groups and control groups are wounded on the dorsal skin with a 6-8 mm diameter punch. Kinetics of wound closure is evaluated by measuring the wound size over time. A subset of mice from each group is sacrificed every 2 days for 20 days, and the wound sites are collected for histological analysis of senescence by SA-β-GAL staining and gene expression analysis to determine the presence of senescence cells by p16 expression, markers of the senescence-associated secretory phenotype (SASP) such as 1IL-1, IL-6, CXCL-1 and MMP3, and immune cell infiltration by IHC targeting the pan leucocyte marker CD45.

### Example 18

### Assessing the Impact of Decreased FOXO4 Activity on Improvement of Symptoms Of Diseases Associated with Kyphosis

Kyphosis is a severe curvature in the spinal column, and it is frequently seen with normal and premature aging (Katzman et al. (2010) “Age-related hyperkyphosis: its causes, consequences, and management.” J Orthop Sports Phys Ther 40: 352-360). Kyphosis has been associated with cellular senescence. TTD mice, which are described in Example 14, are known to develop kyphosis (de Boer et al. (2002) “Premature aging in mice deficient in DNA repair and transcription.” Science 296: 1276-1279).

To assess the role of FOXO4 in kyphosis, a first group of TTD mice is infected with FOXO4 shRNA lentivirus, such as those described above, and a second group of TTD mice is treated with FOXO4-p53 blocking peptides, such as those described above. A third group of TTD mice receives neither virus nor peptide. Kyphosis formation is visually measured in time in the test groups of mice and compared to a control groups of syngeneic mice that do not carry the TTD mutation. Post mortem, mice from the test groups and control groups are analyzed for the presence of senescent cells by SA-β-Gal staining.

BubR1^{H/H} mice, which are described in Example 15, are also known to develop kyphosis (Baker et al. (2011) “Clearance of p16Ink4a-positive senescent cells delays ageing-associated disorders.” Nature 479: 232-236). Thus, the experiment described above is also performed in BubR1^{H/H} mice.

### Example 19

### Assessing the Effects of Decreased FOXO4 Activity on the Symptoms of Osteoarthritis

Osteoarthritis is a degenerative joint disease. With increasing age, the prevalence of osteoarthritis increases, and the efficacy of articular cartilage repair decreases. These effects have been associated with increased senescence (Martin et al. (2003) “The role of chondrocyte senescence in the pathogenesis of osteoarthritis and in limiting cartilage repair.” J Bone Joint Surg Am 85-A Suppl 2: 106-110). TTD mice, which are described in Example 14, are a validated animal model for the study of osteoarthritis (Botter et al. (2011) “Analysis of osteoarthritis in a mouse model of the progeroid human DNA repair syndrome trichothiodystrophy.” Age (Dordr) 33: 247-260).

To assess the role of FOXO4 in osteoarthritis, a first group of TTD mice is transduced with FOXO4 shRNA viruses, such as those described above, and a second group of is treated with FOXO4-p53 blocking peptides, such as those described above. A third group of TTD mice received neither virus nor peptide treatment. The rate of calcification of the proximal portion of the tibiae is observed via micro-CT in the test groups, as described (Botter, S. M., G. J. van Osch, et al. (2006). “Quantification of subchondral bone changes in a murine osteoarthritis model using micro-CT.” Biorheology 43(3-4): 379-388) and compared to the rate of calcification in control groups of syngeneic mice that do not carry the TTD mutation. Post mortem, mice from each test group and control group are analyzed for the presence of senescent cells by SA-β-Gal staining.

### Example 20

### Assessing the Effects of Decreased FOXO4 Activity on the Symptoms of Osteoporosis

Age-related osteoblast dysfunction is the main cause of age-related bone loss in both men and women (Kassem et al. (2011) “Senescence-associated intrinsic mechanisms of osteoblast dysfunctions.” Aging Cell 10: 191-197) Impaired osteoblastogenesis has been reported in mouse models for normal aging (Moerman et al. (2004) “Aging activates adipogenic and suppresses osteogenic programs in mesenchymal marrow stroma/stem cells: the role of PPAR-gamma2 transcription factor and TGF-beta/BMP signaling pathways.” Aging Cell 3: 379-389) and senescence-associated accelerated aging (Kajkenova et al. (1997) “Increased adipogenesis and myelopoiesis in the bone marrow of SAMP6, a murine model of defective osteoblastogenesis and low turnover osteopenia.” J Bone Miner Res 12: 1772-1779). TTD mice, which are described above, are a validated model to study osteoporosis (van Apeldoorn et al. (2007) “Physicochemical composition of osteoporotic bone in the trichothiodystrophy premature aging mouse determined by confocal Raman microscopy.” J Gerontol A Biol Sci Med Sci 62: 34-40).

To assess the role of FOXO4 in osteoporosis, a first group of TTD mice is transduced with FOXO4 shRNA viruses, such as those described above, and a second group of TTD mice is treated with FOXO4-p53 blocking peptides, such as those described above. A third group of TTD mice is given neither virus nor peptide. Osteoporosis is visually measured, as described (van Apeldoorn et al. (2007). “Physicochemical composition of osteoporotic bone in the trichothiodystrophy premature aging mouse determined by confocal Raman microscopy.” J Gerontol A Biol Sci Med Sci 62: 34-40) in mice from the test groups and in control groups of syngeneic mice that do not carry the TTD mutation. Post mortem, mice from each test group and control group are analyzed for the presence of senescent cells by SA-β-Gal staining.

### Example 21

### Assessing the Effects of Decreased FOXO4 Activity on the Symptoms of Parkinson's Disease

Parkinson's disease (PD) is the second most common neurodegenerative disease. PD is characterized by slowness of movement (bradykinesia), shaking, stiffness, and loss of balance. A link between senescence and Parkinson's disease has been suggested (Campisi (2011) “Cellular senescence: a link between cancer and age-related degenerative disease?” Semin Cancer Biol 21: 354-359). Experiments are performed to determine whether killing senescent cells by decreasing FOXO4 activity is a suitable method for treatment or prevention of Parkinson's disease.

One culture of healthy human fibroblasts (IMR90) is stably transduced with a FOXO4 short hairpin RNA (shRNA)-expressing virus, such as those described above, and a second culture of IMR90 fibroblasts is exposed to the p53-FOXO4 blocking peptides, such as those described above. A third culture of IMR90 fibroblasts is neither transduced nor exposed to peptides. A PD-like state is induced in the three cell cultures by exposure of the cells to 1-methyl-4-phenyl-1,2,3,6-tetrahydropyridine (MPTP), a mitochondrial complex I inhibitory drug commonly used in vitro and in vivo to induce PD-symptoms (Ferrante et al. (1997) “Systemic administration of rotenone produces selective damage in the striatum and globus pallidus, but not in the substantia nigra.” Brain Res 753: 157-162).

The percentage of senescence, apoptosis, and oxidation in the test cultures is compared to control cultures of human fibroblasts in which PD is not induced. The percentage senescence is scored by SA-β-GAL assay and the percentage apoptosis in time is scored by TUNEL staining. In addition, the level of cellular oxidation is determined by Redox-Maleimide technique (Mastroberardino et al. (2008) “A FRET-based method to study protein thiol oxidation in histological preparations.” Free Radic Biol Med 45: 971-981).

To assess the role of FOXO4 in fibroblasts derived from PD patients, one culture of PD-derived fibroblasts is stably transduced with a FOXO4 short hairpin RNA (shRNA)-expressing virus, such as those described above, and a second culture of PD-derived fibroblasts is exposed to the p53-FOXO4 blocking peptides, such as those described above. A third culture of fibroblasts from PD-patients is neither transduced nor exposed to peptides.

Similarly, one culture of fibroblasts derived from age-matched healthy controls is stably transfected with FOXO4 short hairpin RNA (shRNA)-expressing virus, and a second culture is exposed to the p53-FOXO4 blocking peptides. A third culture of fibroblasts derived from age-matched healthy controls is neither transfected nor exposed to blocking peptides.

All six cultures are exposed to senescence-inducing levels of ionizing radiation, and cultures of cells are assessed to determine percentage of apoptosis, percentage of senescence, and oxidation, as described above.

The Rotarod method measures how long a mouse remains on a rotating beam (measured as latency to fall). Mice with motor deficits, such as MPTP-treated mice, fall off more quickly than mice with no motor deficits (Meredith et al. (2008) “Animal models of Parkinson's disease progression.” Acta Neuropathol 115: 385-398). To assess the role of FOXO4 in PD-like disease in vivo, one group of healthy mice is pre-treated with FOXO4 shRNA viruses, such as those described above, and a second group of healthy mice is given FOXO4-p53 blocking peptides, such as those described above. A third group of healthy mice is given neither virus nor blocking peptides. The three groups of mice are exposed to MPTP. The motor performances of the three groups of mice is analyzed by Rotarod assay and compared to the motor performance of control groups of mice that were not exposed to MPTP. As PD is characterized by the death of dopaminergic neurons in the substantia nigra, mice from each test and control group are sacrificed, and their tissues are assayed for senescence, apoptosis and oxidation of Substantia Nigra as described (Kim et al. (2003) “Parkin cleaves intracellular alpha-synuclein inclusions via the activation of calpain.” J Biol Chem 278: 41890-41899).

### Example 22

### Assessing the Impact of Decreased FOXO4 Activity on Improvement of Symptoms of Diseases Associated with Renal Dysfunction

Many nephrological pathologies arise in the elderly, and one such pathology is Glomerular Disease (GD). Some subtypes of GD, such as glomerulonephritis, are characterized by inflammation of the kidney and by the expression of two proteins, IL1α and IL1β (Niemir, Z. I., H. Stein, et al. (1997). “Podocytes are the major source of IL-1 alpha and IL-1 beta in human glomerulonephritides.” Kidney Int 52: 393-403). IL1α and IL1β are master regulators of the senescence-associated secretory phenotype (SASP) (Coppe et al. (2008) “Senescence-associated secretory phenotypes reveal cell-nonautonomous functions of oncogenic RAS and the p53 tumor suppressor.” PLoS. Biol. 6: 2853-2868). GD is associated with elevated presence of senescent cells, especially in fibrotic kidneys (Sis et al. (2007) “Accelerated expression of senescence associated cell cycle inhibitor p161NK4A in kidneys with glomerular disease.” Kidney Int 71: 218-226).

Renal failure following kidney transplantation is another kidney-related pathology associated with senescence. The cause of renal failure following allograft transplantations in humans and mice is ischemia-reperfusion. Renal failure following allograft transplantations is also observed more frequently with kidneys from older donors (Naesens et al. (2009). “Donor age and renal P-glycoprotein expression associate with chronic histological damage in renal allografts.” J Am Soc Nephrol 20: 2468-2480). This is thought to be caused by a marked increase in senescence as a consequence of the ischemia-reperfusion response (Braun et al. (2012) “Cellular senescence limits regenerative capacity and allograft survival.” J Am Soc Nephrol 23:1467-1473). As FOXO4 inhibition induces an apoptosis response in senescent kidney tissue, the effects of FOXO4 inhibition on the symptoms of renal diseases are assessed.

Glomerular disease is studied in mouse models. For example, mice carrying a mutation in Atg5, a protein involved with autophagy, are susceptible to spontaneous glomerular disease with age (Hartleben et al. (2010) “Autophagy influences glomerular disease susceptibility and maintains podocyte homeostasis in aging mice.” J Clin Invest 120: 1084-1096). Senescence is linked to autophagy (Narita et al. (2009) “Autophagy facilitates oncogene-induced senescence.” Autophagy 5: 1046-1047).

One group of Atg5^{-/-} mice is pre-treated with FOXO4 shRNA viruses, such as those described above, and a second group of Atg5^{-/-} mice is given FOXO4-p53 blocking peptides, such as those described above. A third group of Atg5_{-/-} mice is given neither virus nor blocking peptides. Kidney function is measured in mice from each test group as described (Susa, et al. (2009) “Congenital DNA repair deficiency results in protection against renal ischemia reperfusion injury in mice.” Aging Cell 8:192-200) and compared to kidney function in control groups of syngeneic mice that do not carry the Atg5 mutation. Post mortem, mice from each test group and control group are analyzed for the presence of senescent cells by SA-β-Gal staining.

BAFF overexpression is associated with glomerular pathology in mice (Stohl et al. (2005) “BAFF overexpression and accelerated glomerular disease in mice with an incomplete genetic predisposition to systemic lupus erythematosus.” Arth Rheum. 7: 2080-2091). Accordingly, the experiments described above are repeated in BAFF-overexpressing mice.

Ischemia Reperfusion (IR) following renal transplantation is surgically induced in wildtype mice (Susa et al. (2009). “Congenital DNA repair deficiency results in protection against renal ischemia reperfusion injury in mice.” Aging Cell 8: 192-200). To assess the role of FOXO4 in IR following renal transplant, a first group of mice is transduced with FOXO4 shRNA viruses, such as those described above, and a second group of mice is treated with FOXO4-p53 blocking peptides, such as those described above. A third group of mice is given neither virus nor peptide. IR is then surgically induced in mice from each of the three groups, and kidney function in these mice is assayed as described above.

### Example 23

### Assessing the Impact of Decreased FOXO4 Activity on Improvement of Symptoms of Herniated Intervertebral Discs

Patients with herniated intervertebral discs exhibit elevated presence of cell senescence in the blood and in vessel walls (Roberts et al. (2006) “Senescence in human intervertebral discs.” Eur Spine J 15 Suppl 3: S312-316). Increased levels of proinflammatory molecules and matrix metalloproteases are also found in aging and degenerating discs tissues, suggesting a role for senescence cells (Chang-Qing et al. (2007) “The cell biology of intervertebral disc aging and degeneration.” Ageing Res Rev. 6: 247-61).

To assess the role of FOXO4 in herniated intervertebral discs, degeneration of the intervertebral disc is induced in mice by compression, as previously described (Lotz et al. (1998) “Compression-induced degeneration of the intervertebral disc: an in vivo mouse model and finite-element study.” Spine (Phila Pa 1976). 23: 2493-506). One group of mice with degenerated vertebral discs are transduced with FOXO4 shRNA viruses, such as those described above, and a second group of mice with degenerated discs are treated with FOXO4-p53, such as those described above. A third group of mice with degenerated discs are given neither virus nor peptide. Disc strength is evaluated in the three test groups of mice and in control groups of mice in which disc degeneration is not induced. The evaluation is performed as described in Lotz et al. (1998) “Compression-induced degeneration of the intervertebral disc: an in vivo mouse model and finite-element study.” Spine (Phila Pa 1976). 23: 2493-506. Post mortem, mice from each test group and control group are analyzed for the presence of senescent cells by SA-β-Gal staining.

Although the foregoing invention has been described in some detail by way of illustration and example for purposes of clarity of understanding, the descriptions and examples should not be construed as limiting the scope of the invention.

What is claimed is:

1. A method of treating cancer in an individual comprising administering to the individual an effective amount of an agent that inhibits FOXO4, wherein the agent is used as an adjuvant therapy.

2. The method of claim 1, wherein the agent is a peptide that inhibits FOXO4 function in a cell, wherein the peptide comprises an amino acid sequence that has at least 80% identity to a fragment of the FOXO4.

3. The method of claim 1, wherein the agent is a peptide comprising an amino acid sequence that has at least 80% identity to a fragment in the DNA binding domain or C-terminal region of FOXO4.

4. An isolated peptide that inhibits FOXO4 function in a cell, wherein the peptide comprises an amino acid sequence that has at least 80% identity to a fragment of the FOXO4.

5. The peptide of claim 4, wherein the peptide comprises an amino acid sequence that has at least 80% identity to a fragment in the DNA binding domain of FOXO4.

6. The peptide of claim 4, wherein the peptide comprises an amino acid sequence that has at least 80% identity to a fragment in the C-terminal region of FOXO4.

7. The peptide of claim 4, wherein the peptide comprises the amino acid sequence selected from the group consisting of (i) SRRNAWGNQSYAELIS, (ii) PRKGGSRRNAWGNQSYAELISQAIESAPEKRLTL; (iii) LECDMDNIISDLMDEGEGLDF; and (iv) PQDLDLDMYMENLECDMDNIISDLMDEGEGLDF.

8. The peptide of claim 4, wherein the peptide further comprises an amino acid sequence that facilitates entry into a cell.

9. The peptide of claim 8, wherein the sequence that facilitates entry into a cell comprises the amino acid sequence GRKKRRQRRR or GRKKRRQRRRPP.

10. A method of treating cancer in an individual comprising administering to the individual an effective amount of the peptide of claim 4.

11. The method of claim 10, wherein the peptide is used as an adjuvant therapy.

12. A method of conferring sensitivity to chemotherapy in cancer cells comprising contacting the cancer cells with an effective amount of the peptide of claim 4.

13. A method of inducing apoptosis of senescent cells comprising contacting the cells with an effective amount of the peptide of claim 4.

14. A method of treating cancer in an individual comprising administering to the individual an effective amount of (a) a peptide comprising an amino acid sequence that has at least 80% identity to a fragment in the DNA binding domain of FOXO4; and/or (b) a peptide comprising an amino acid sequence that has at least 80% identity to a fragment in the C-terminal region of FOXO4.
